(19) United States
(12) Patent Application Publication
(10) Pub. No.: US 2025/0295358 A1
(43) Pub. Date: Sep. 25, 2025

(54) USER INTERFACES FOR MANAGING HEALTH DATA

(71) Applicant: Apple Inc., Cupertino, CA (US)

(72) Inventors: Tito L. BALSAMO, San Francisco, CA (US); Elizabeth LYTLE, Morgan Hill, CA (US); Allison M. STYER, San Francisco, CA (US)

(21) Appl. No.: 19/231,382

(22) Filed: Jun. 6, 2025

Related U.S. Application Data
(63) Continuation of application No. 17/398,810, filed on Aug. 10, 2021.
(60) Provisional application No. 63/064,384, filed on Aug. 11, 2020.
(60) Provisional application No. 63/197,459, filed on Jun. 6, 2021.

(51) Int. Cl. A61B 5/00

(52) U.S. Cl. CPC A61B 5/4815; A61B 5/4809; A61B 5/4812; A61B 5/743; A61B 5/7435

(57) ABSTRACT

The present disclosure generally relates to managing health data for a patient. In some embodiments, the disclosed techniques include displaying graphical representations of data including a first graph corresponding to a first data set and a second graph corresponding to a second data set. An input directed to the first graph is detected, and in response, a plurality of user interface objects are displayed, including a first user interface object or a second user interface object. The first user interface object is associated with the first graph and based on a first variable that is selected based on a location of the input, and the second user interface object is associated with the second graph and based on a second variable that is selected based on the location of the first input. In some embodiments, the health data is sleep-related data, including data on mid-sleep awakenings.

### CROSS-REFERENCE TO RELATED APPLICATIONS

This application is a continuation of U.S. patent application Ser. No. 17/398,810, entitled “USER INTERFACES FOR MANAGING HEALTH DATA,” filed on Aug. 10, 2021, which claims priority to U.S. Provisional Patent Application No. 63/197,459, entitled “USER INTERFACES FOR MANAGING HEALTH DATA,” filed on Jun. 6, 2021, and U.S. Provisional Patent Application No. 63/064,384, entitled “USER INTERFACES FOR MANAGING HEALTH DATA,” filed Aug. 11, 2020. The contents of the aforementioned applications are hereby incorporated by reference in their entireties.

### FIELD

The present disclosure relates generally to computer user interfaces, and more specifically to techniques for managing health data.

### BACKGROUND

An electronic device can be used to manage health data for a patient. Information concerning health data can be presented to a user on the electronic device.

### BRIEF SUMMARY

Some techniques for managing health data for a patient using electronic devices are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for managing, interacting with, and visualizing health data for a patient, including interrelated or potentially interrelated data. Such methods and interfaces optionally complement or replace other methods for managing health data for a patient. Such methods and interfaces reduce the cognitive burden on a user and produce a more efficient human-machine interface. For battery-operated computing devices, such methods and interfaces conserve power and increase the time between battery charges.

In accordance with some embodiments, a method performed at a computer system that is in communication with a display generation component and one or more input devices is described. The method comprises: displaying, via the display generation component, a plurality of graphical representations of data, including: a first graphical representation of data corresponding to a first data set; and a second graphical representation of data corresponding to a second data set that is different from the first data set; detecting, via the one or more input devices, a first input corresponding to the first graphical representation of data; and in response to detecting the first input corresponding to the first graphical representation of data, displaying a plurality of user interface objects, including: a first user interface object associated with the first graphical representation of data and based on a first variable that is selected based on a location of the first input, including: in accordance with a determination that the first input corresponds to a first location in the first graphical representation of data, a representation of a first subset of the first data set associated with the first variable; and in accordance with a determination that the first input corresponds to a second location in the first graphical representation of data different from the first location, a representation of a second subset of the first data set that is associated with the first variable and that is different than the first subset of the first data set; and a second user interface object associated with the second graphical representation of data and based on a second variable that is selected based on a location of the first input, including: in accordance with a determination that the first input corresponds to the first location in the first graphical representation of data, a representation of a first subset of the second data set associated with the second variable; and in accordance with a determination that the first input corresponds to the second location in the first graphical representation of data different from the first location, a representation of a second subset of the second data set that is associated with the second variable and that is different than the first subset of the second data set.

In accordance with some embodiments, a non-transitory computer-readable storage medium storing one or more programs configured to be executed by one or more processors of a computer system in communication with a display generation component and one or more input devices is described. The one or more programs include instructions for: displaying, via the display generation component, a plurality of graphical representations of data, including: a first graphical representation of data corresponding to a first data set; and a second graphical representation of data corresponding to a second data set that is different from the first data set; detecting, via the one or more input devices, a first input corresponding to the first graphical representation of data; and in response to detecting the first input corresponding to the first graphical representation of data, displaying a plurality of user interface objects, including: a first user interface object associated with the first graphical representation of data and based on a first variable that is selected based on a location of the first input, including: in accordance with a determination that the first input corresponds to a first location in the first graphical representation of data, a representation of a first subset of the first data set associated with the first variable; and in accordance with a determination that the first input corresponds to a second location in the first graphical representation of data different from the first location, a representation of a second subset of the first data set that is associated with the first variable and that is different than the first subset of the first data set; and a second user interface object associated with the second graphical representation of data and based on a second variable that is selected based on a location of the first input, including: in accordance with a determination that the first input corresponds to the first location in the first graphical representation of data, a representation of a first subset of the second data set associated with the second variable; and in accordance with a determination that the first input corresponds to the second location in the first graphical representation of data different from the first location, a representation of a second subset of the second data set that is associated with the second variable and that is different than the first subset of the second data set.

In accordance with some embodiments, a transitory computer-readable storage medium storing one or more programs configured to be executed by one or more processors of a computer system in communication with a display generation component and one or more input devices is described. The one or more programs include instructions for: displaying, via the display generation component, a plurality of graphical representations of data, including: a first graphical representation of data corresponding to a first data set; and a second graphical representation of data corresponding to a second data set that is different from the first data set; detecting, via the one or more input devices, a first input corresponding to the first graphical representation of data; and in response to detecting the first input corresponding to the first graphical representation of data, displaying a plurality of user interface objects, including: a first user interface object associated with the first graphical representation of data and based on a first variable that is selected based on a location of the first input, including: in accordance with a determination that the first input corresponds to a first location in the first graphical representation of data, a representation of a first subset of the first data set associated with the first variable; and in accordance with a determination that the first input corresponds to a second location in the first graphical representation of data different from the first location, a representation of a second subset of the first data set that is associated with the first variable and that is different than the first subset of the first data set; and a second user interface object associated with the second graphical representation of data and based on a second variable that is selected based on a location of the first input, including: in accordance with a determination that the first input corresponds to the first location in the first graphical representation of data, a representation of a first subset of the second data set associated with the second variable; and in accordance with a determination that the first input corresponds to the second location in the first graphical representation of data different from the first location, a representation of a second subset of the second data set that is associated with the second variable and that is different than the first subset of the second data set.

In accordance with some embodiments, a computer system in communication with a display generation component and one or more input devices is described. The computer system in communication with a display generation component and one or more input devices comprises one or more processors, and memory storing one or more programs configured to be executed by the one or more processors. The one or more programs include instructions for: displaying, via the display generation component, a plurality of graphical representations of data, including: a first graphical representation of data corresponding to a first data set; and a second graphical representation of data corresponding to a second data set that is different from the first data set; detecting, via the one or more input devices, a first input corresponding to the first graphical representation of data; and in response to detecting the first input corresponding to the first graphical representation of data, displaying a plurality of user interface objects, including: a first user interface object associated with the first graphical representation of data and based on a first variable that is selected based on a location of the first input, including: in accordance with a determination that the first input corresponds to a first location in the first graphical representation of data, a representation of a first subset of the first data set associated with the first variable; and in accordance with a determination that the first input corresponds to a second location in the first graphical representation of data different from the first location, a representation of a second subset of the first data set that is associated with the first variable and that is different than the first subset of the first data set; and a second user interface object associated with the second graphical representation of data and based on a second variable that is selected based on a location of the first input, including: in accordance with a determination that the first input corresponds to the first location in the first graphical representation of data, a representation of a first subset of the second data set associated with the second variable; and in accordance with a determination that the first input corresponds to the second location in the first graphical representation of data different from the first location, a representation of a second subset of the second data set that is associated with the second variable and that is different than the first subset of the second data set.

In accordance with some embodiments, a computer system in communication with a display generation component and one or more input devices is described. The computer system in communication with a display generation component and one or more input devices comprises means for displaying, via the display generation component, a plurality of graphical representations of data, including: a first graphical representation of data corresponding to a first data set; and a second graphical representation of data corresponding to a second data set that is different from the first data set; means for detecting, via the one or more input devices, a first input corresponding to the first graphical representation of data; and means for, in response to detecting the first input corresponding to the first graphical representation of data, displaying a plurality of user interface objects, including: a first user interface object associated with the first graphical representation of data and based on a first variable that is selected based on a location of the first input, including: in accordance with a determination that the first input corresponds to a first location in the first graphical representation of data, a representation of a first subset of the first data set associated with the first variable; and in accordance with a determination that the first input corresponds to a second location in the first graphical representation of data different from the first location, a representation of a second subset of the first data set that is associated with the first variable and that is different than the first subset of the first data set; and a second user interface object associated with the second graphical representation of data and based on a second variable that is selected based on a location of the first input, including: in accordance with a determination that the first input corresponds to the first location in the first graphical representation of data, a representation of a first subset of the second data set associated with the second variable; and in accordance with a determination that the first input corresponds to the second location in the first graphical representation of data different from the first location, a representation of a second subset of the second data set that is associated with the second variable and that is different than the first subset of the second data set.

In accordance with some embodiments, a method performed at a computer system that is in communication with a display generation component and one or more input devices is described. The method comprises: receiving, via the one or more input devices, a set of sleep data that includes data for a first plurality of sleep sessions of a first user; and after receiving the set of sleep data, displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

In accordance with some embodiments, a non-transitory computer-readable storage medium is described. The non-transitory computer-readable storage medium stores one or more programs configured to be executed by one or more processors of a computer system that is in communication with a display generation component and one or more input devices, the one or more programs including instructions for: receiving, via the one or more input devices, a set of sleep data that includes data for a first plurality of sleep sessions of a first user; and after receiving the set of sleep data, displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

In accordance with some embodiments, a transitory computer-readable storage medium is described. The transitory computer-readable storage medium stores one or more programs configured to be executed by one or more processors of a computer system that is configured to communicate with a display generation component and one or more input devices, the one or more programs including instructions for: receiving, via the one or more input devices, a set of sleep data that includes data for a first plurality of sleep sessions of a first user; and after receiving the set of sleep data, displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

In accordance with some embodiments, a computer system that is configured to communicate with a display generation component and one or more input devices is described. The computer system comprises: one or more processors; and memory storing one or more programs configured to be executed by the one or more processors, the one or more programs including instructions for: receiving, via the one or more input devices, a set of sleep data that includes data for a first plurality of sleep sessions of a first user; and after receiving the set of sleep data, displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

In accordance with some embodiments, a computer system that is configured to communicate with a display generation component and one or more input devices is described. The computer system comprises: means for receiving, via the one or more input devices, a set of sleep data that includes data for a first plurality of sleep sessions of a first user; and means, after receiving the set of sleep data, for displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

In accordance with some embodiments, a computer program product is described. The computer program product comprises one or more programs configured to be executed by one or more processors of a computer system that is in communication with a display generation component and one or more input devices. The one or more programs include instructions for: receiving, via the one or more input devices, a set of sleep data that includes data for a first plurality of sleep sessions of a first user; and after receiving the set of sleep data, displaying, via the display generation component, a sleep analysis user interface that includes: a first sleep indicator that indicates a sleep period for a first sleep session of the first plurality of sleep sessions; in accordance with a determination that the data for the first plurality of sleep sessions includes at least one mid-sleep awakening event corresponding to the first sleep session, a first awakening indicator that indicates a mid-sleep awakening event for the first sleep session; a second sleep indicator that indicates a sleep period for a second sleep session of the first plurality of sleep sessions, different from the first sleep session; in accordance with a determination that the data for the first plurality of sleep sessions includes at least one mid-sleep awakening event corresponding to the second sleep session, a second awakening indicator that indicates a mid-sleep awakening event for the second sleep session; and a collective awakening indicator that indicates a value based on the collective mid-sleep awakening events for the first plurality of sleep sessions.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing health data for a patient, thereby increasing the effectiveness, efficiency, and user satisfaction with such devices. Such methods and interfaces may complement or replace other methods for managing health data for a patient.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6K illustrate exemplary user interfaces for managing health data for a patient, in accordance with some embodiments.

FIGS. 7A and 7B are a flow diagram illustrating a method for managing health data for a patient using a computer system, in accordance with some embodiments.

FIGS. 8A-8G illustrate exemplary user interfaces for managing health data for a patient, specifically sleep-related data, in accordance with some embodiments.

FIGS. 9A-9D illustrate exemplary user interfaces for managing health data for a patient, in accordance with some embodiments.

FIG. 10 is a flow diagram illustrating a method for managing health data for a patient, specifically sleep-related data, using a computer system, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

In some implementations, an example electronic device provides efficient methods and interfaces for managing health data for a patient. For example, the example electronic device can provide a user with information about patient health data in an easily understandable and convenient manner. Such techniques can reduce the cognitive burden on a user who accesses health data for a patient, thereby enhancing productivity. Further, such techniques can reduce processor and battery power otherwise wasted on redundant user inputs.

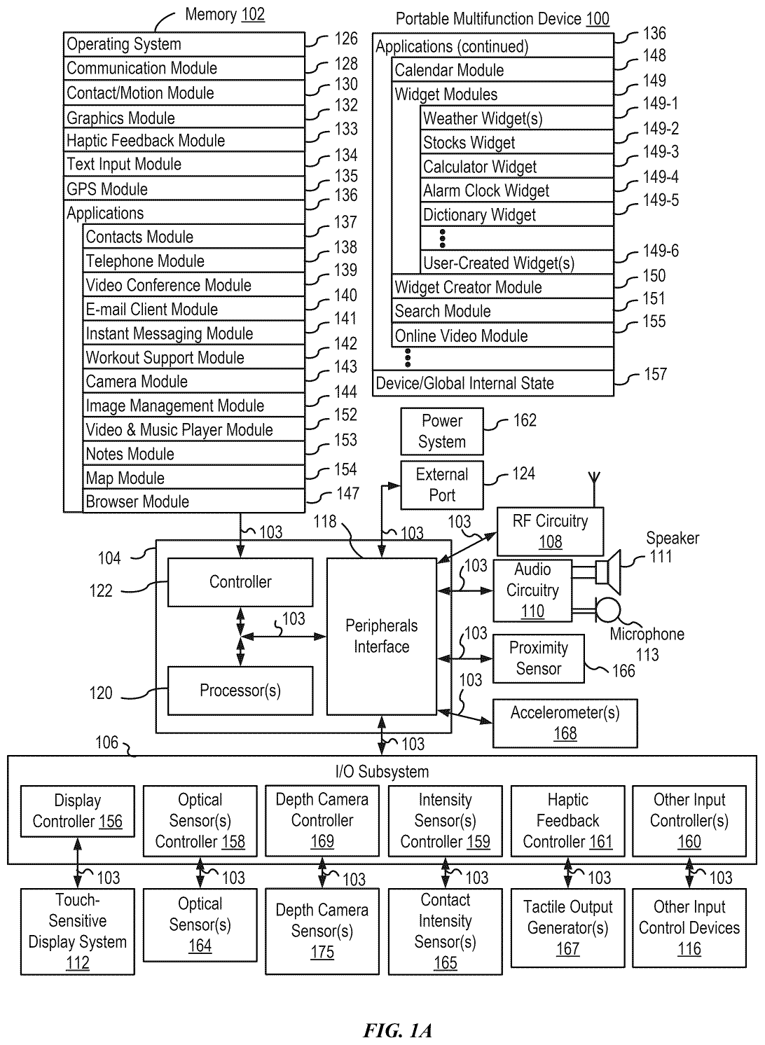

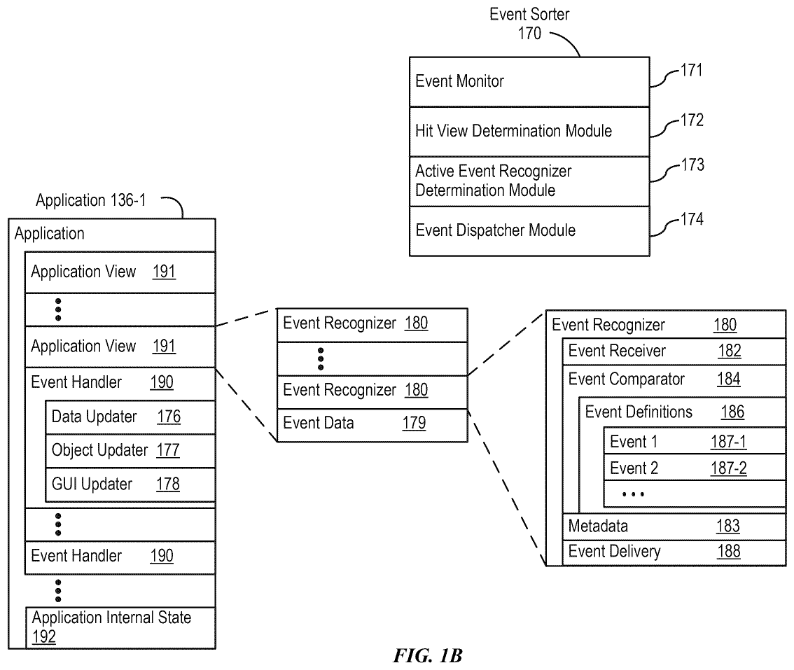

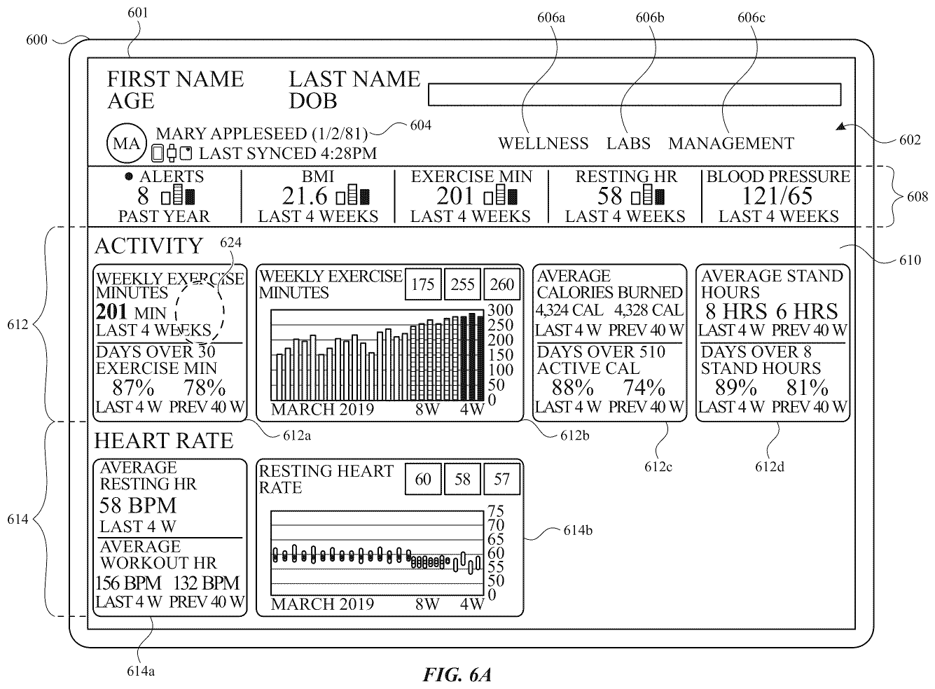

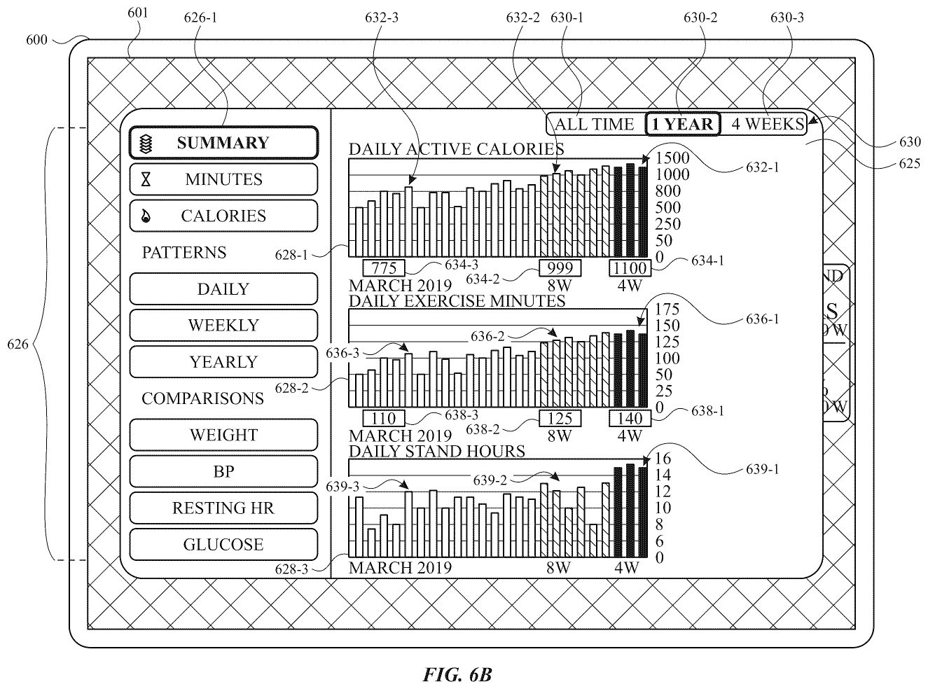

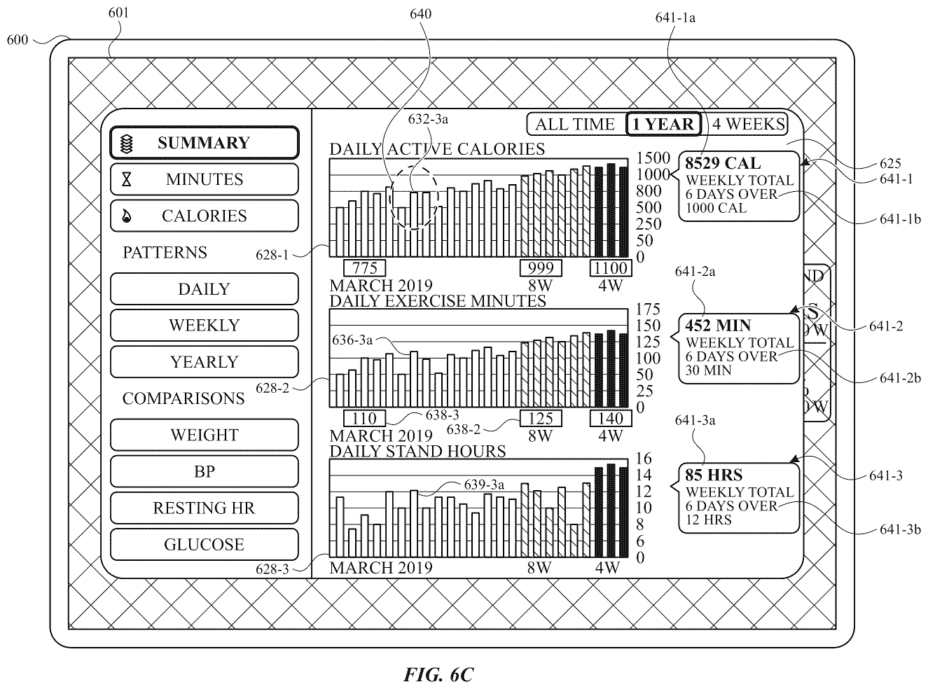

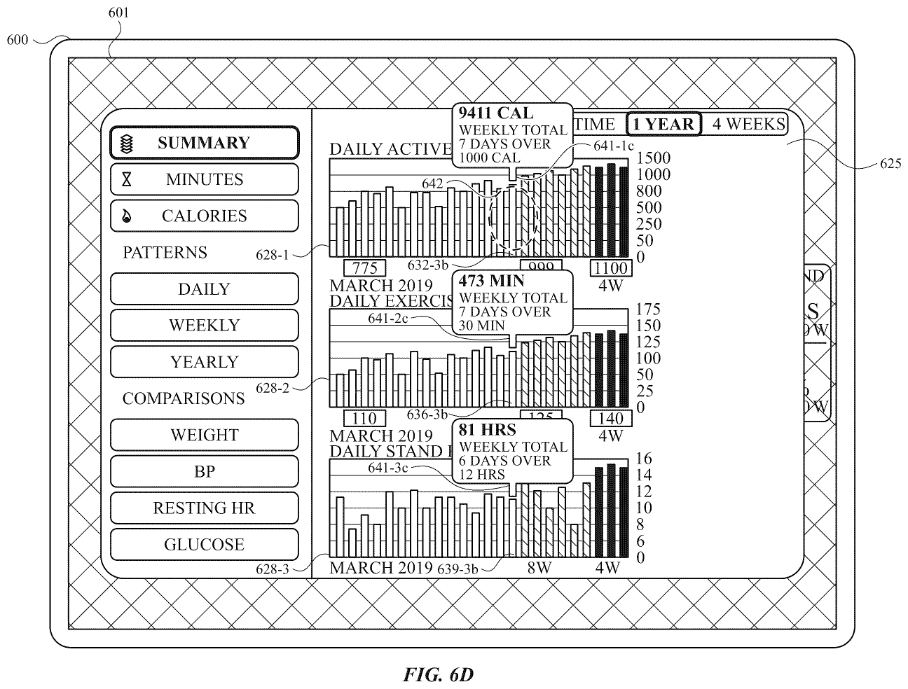

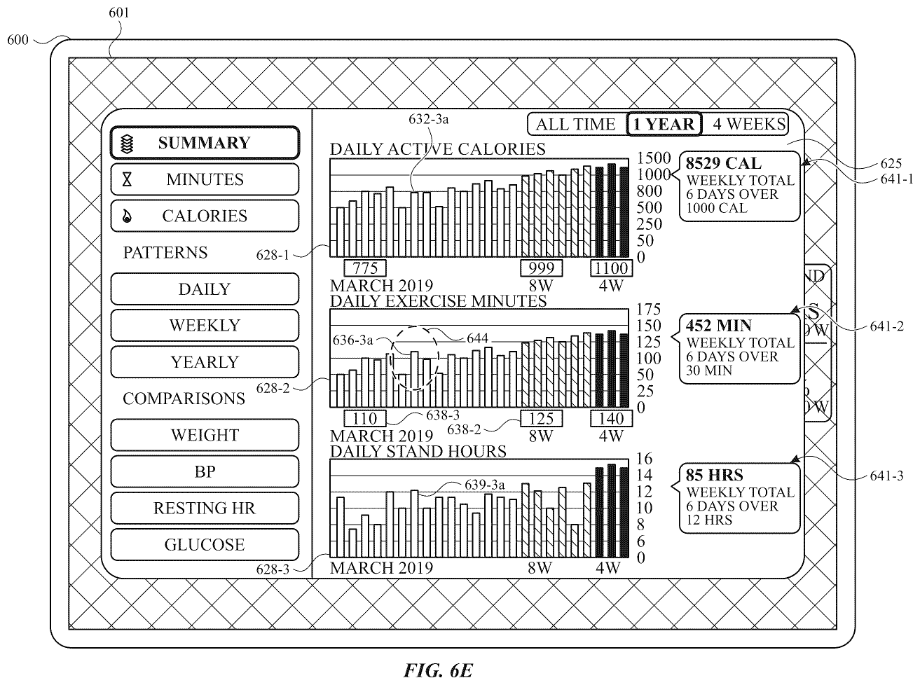

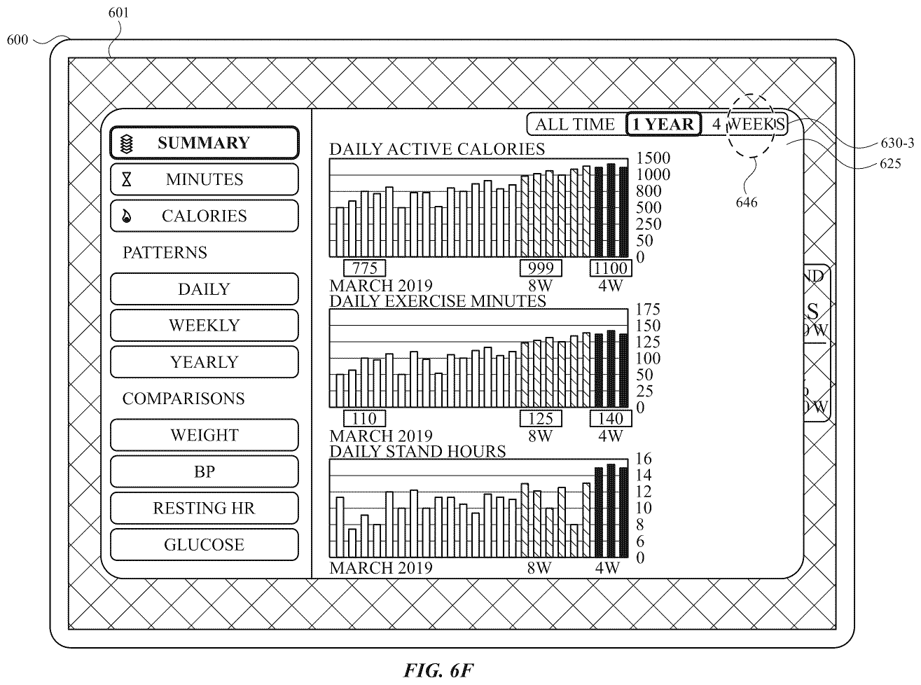

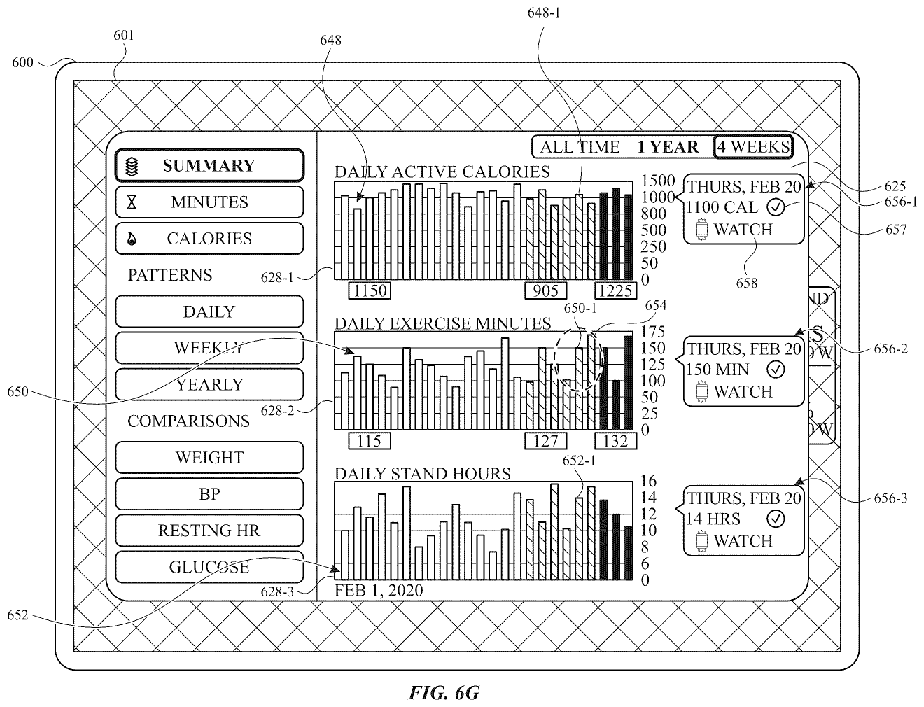

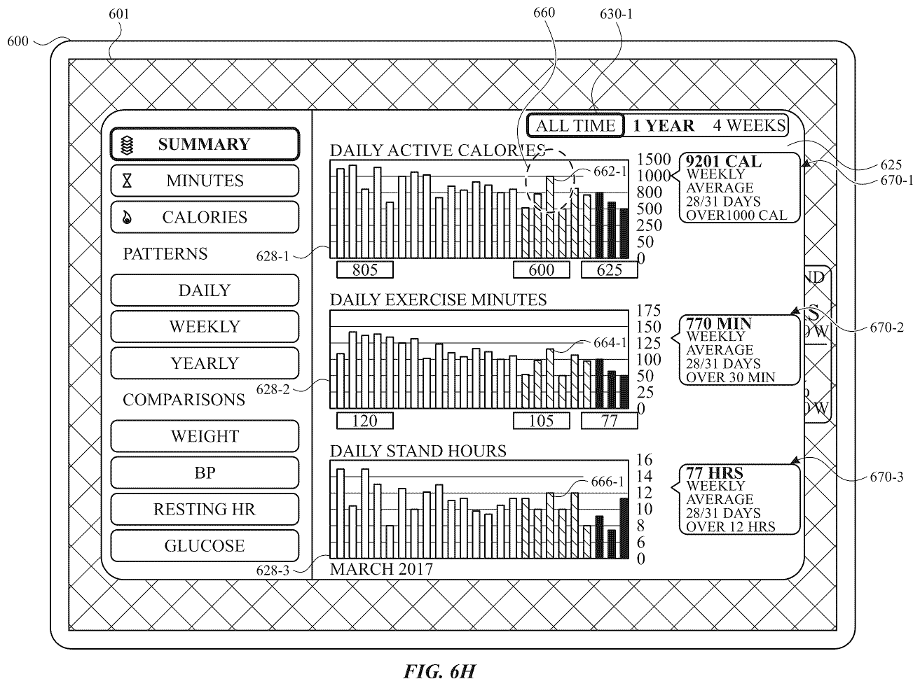

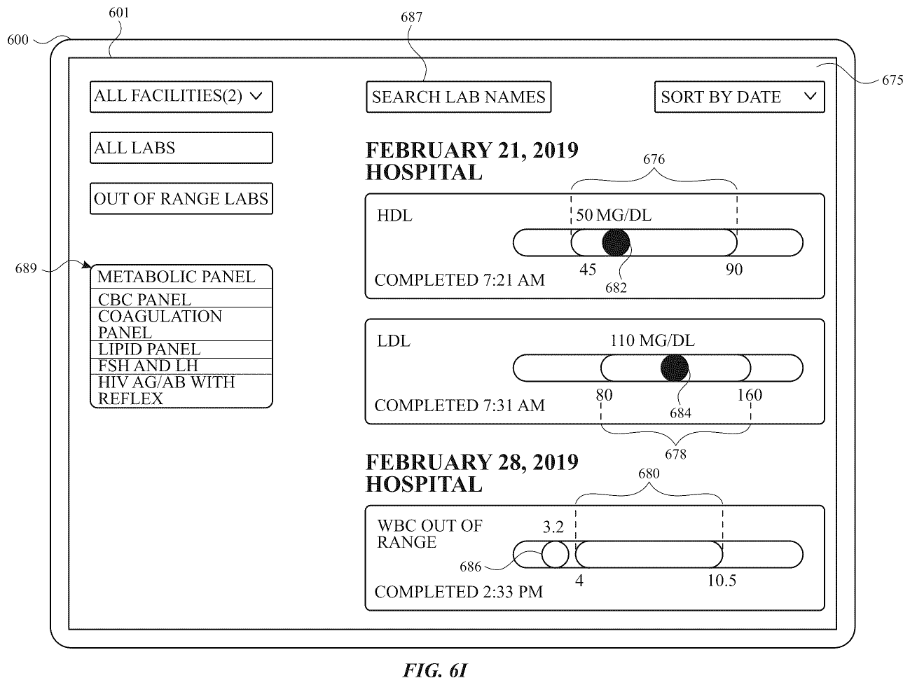

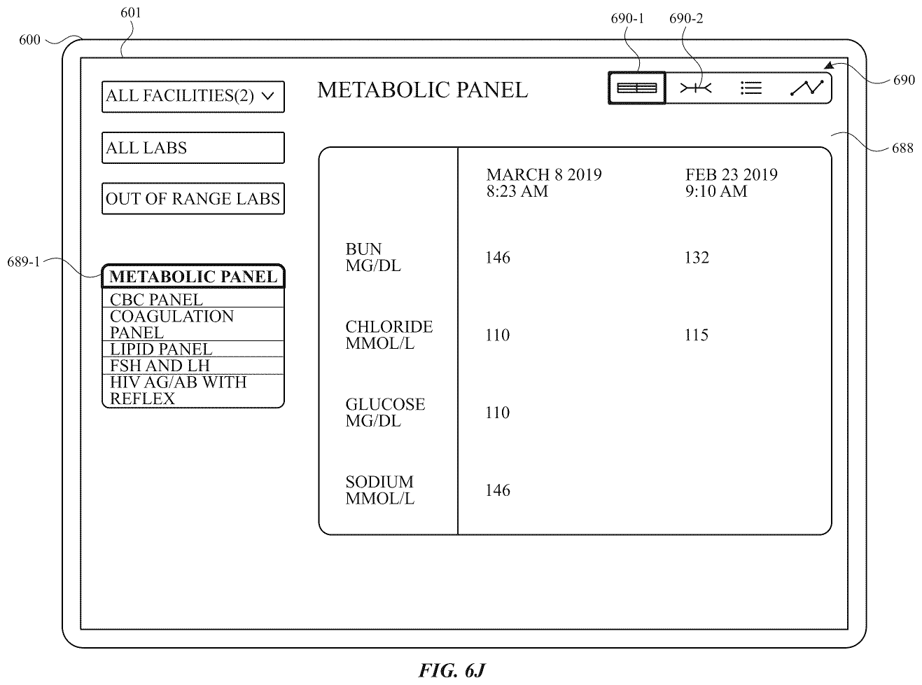

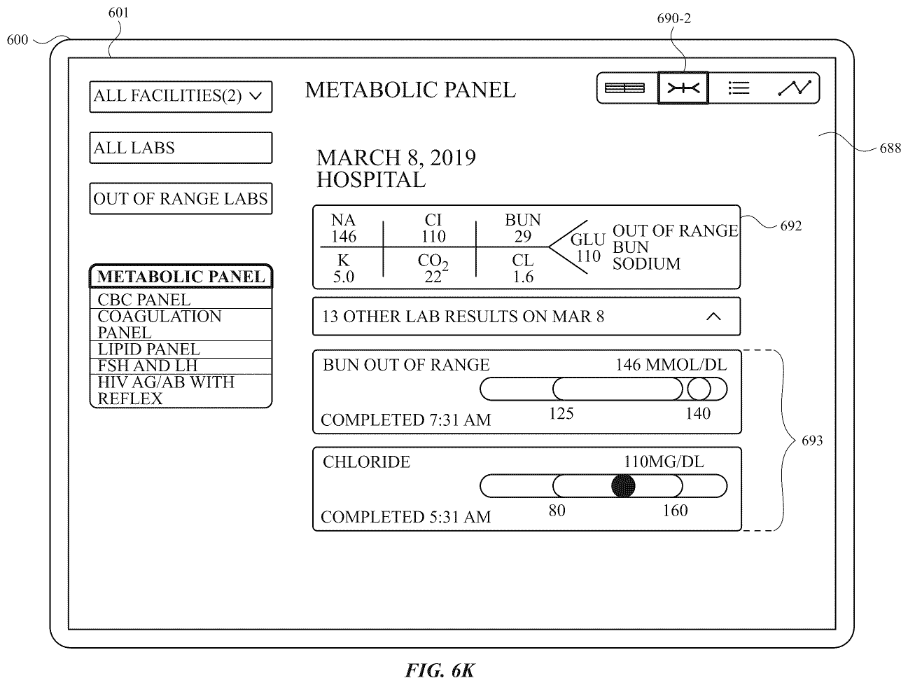

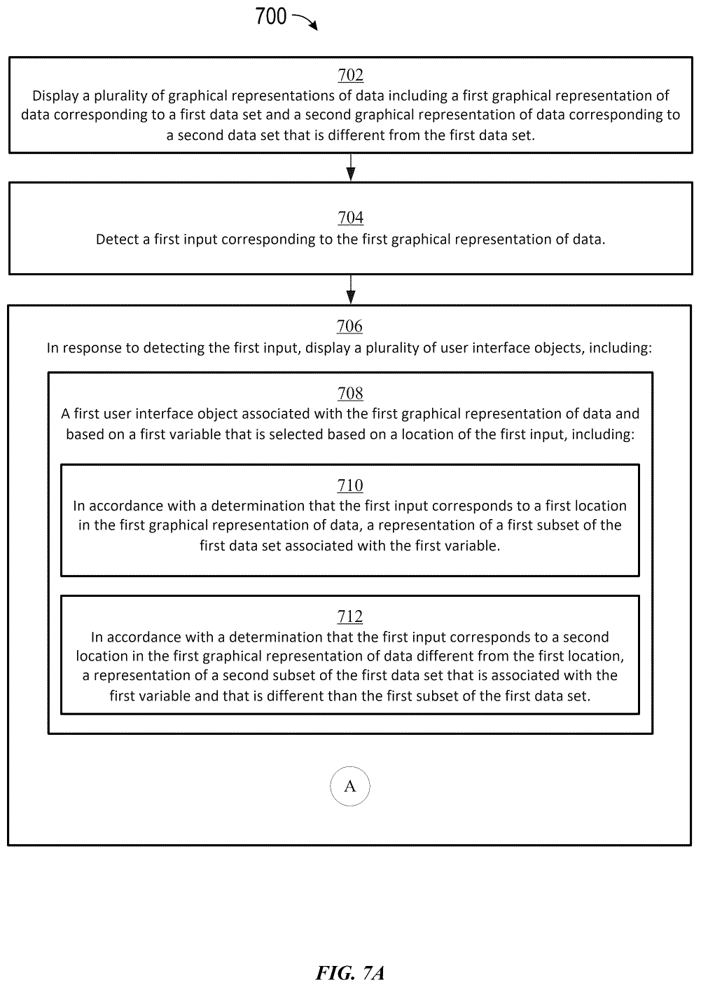

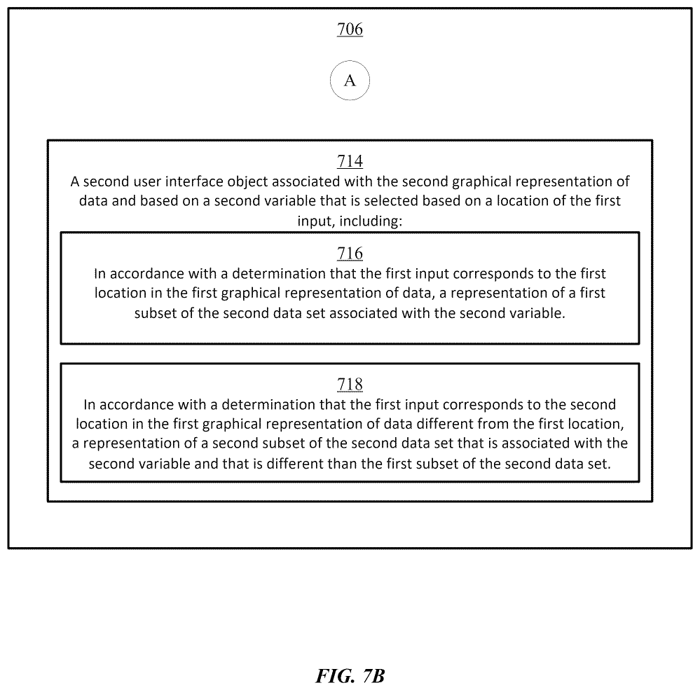

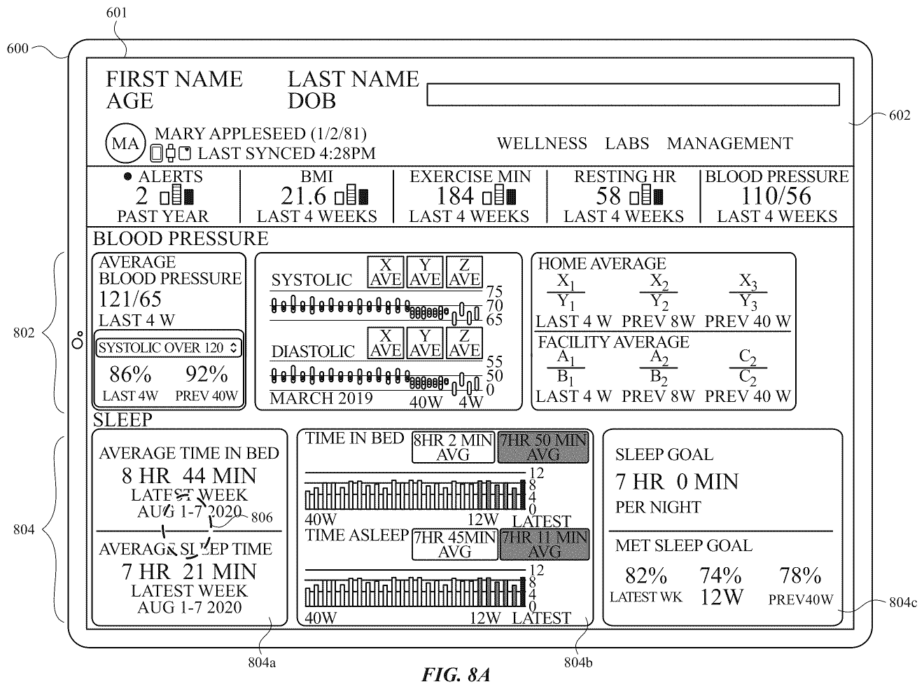

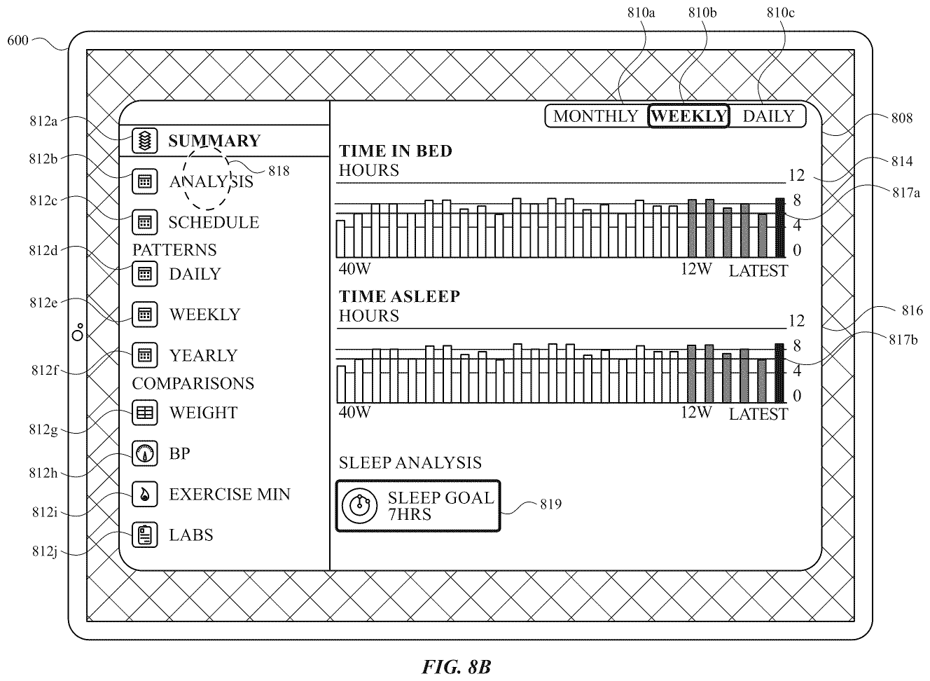

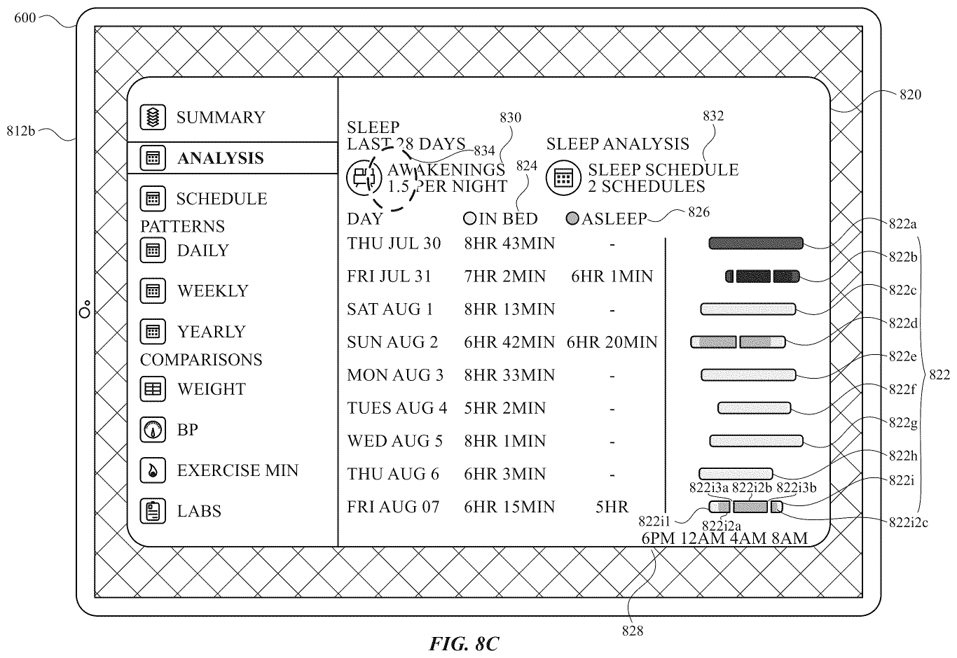

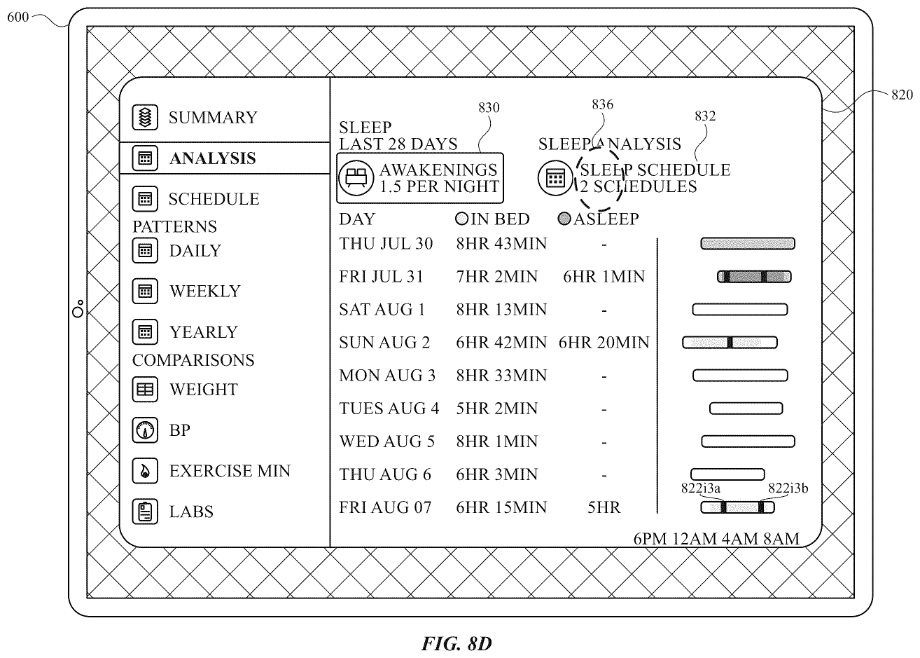

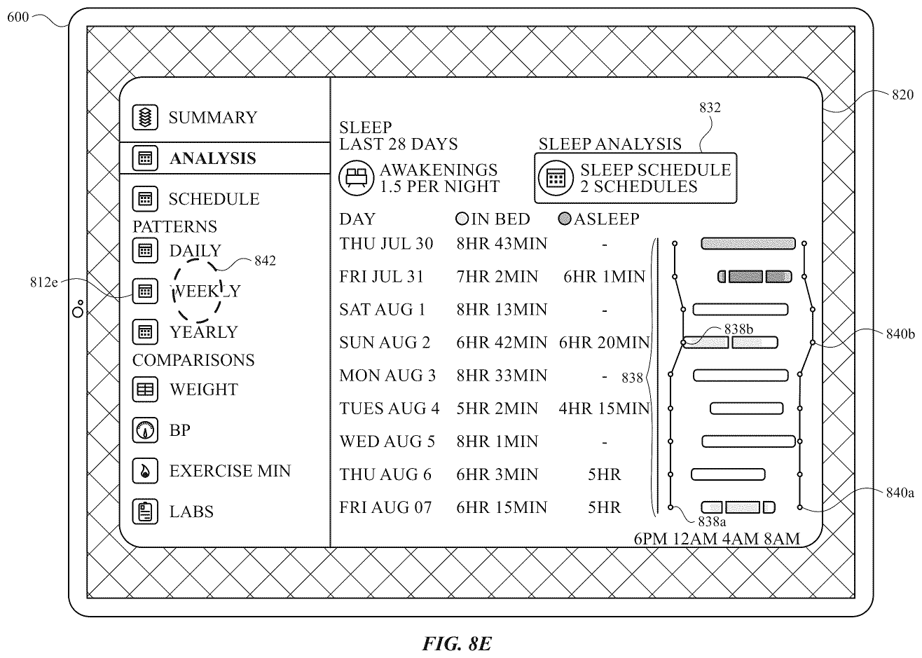

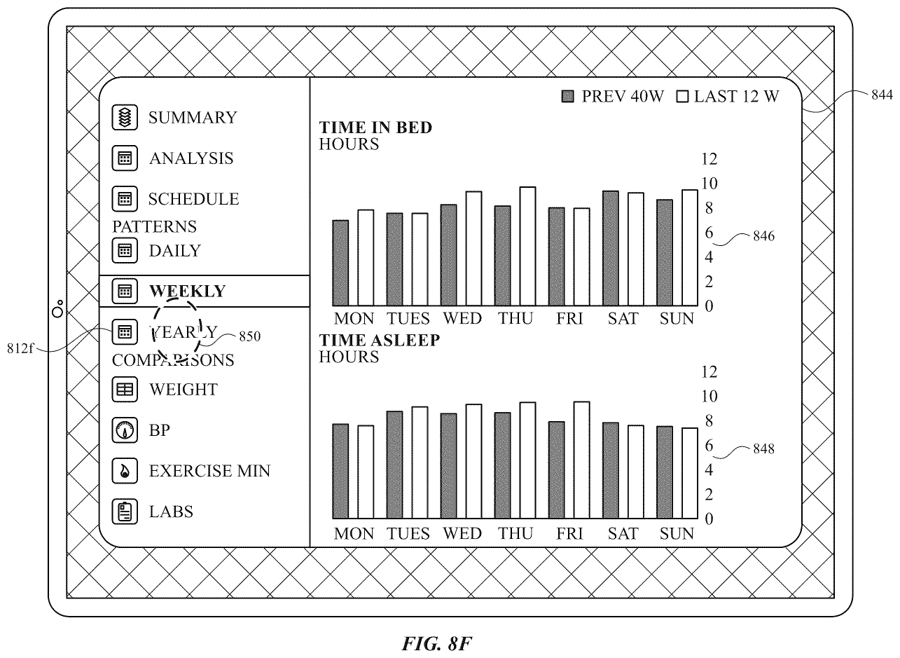

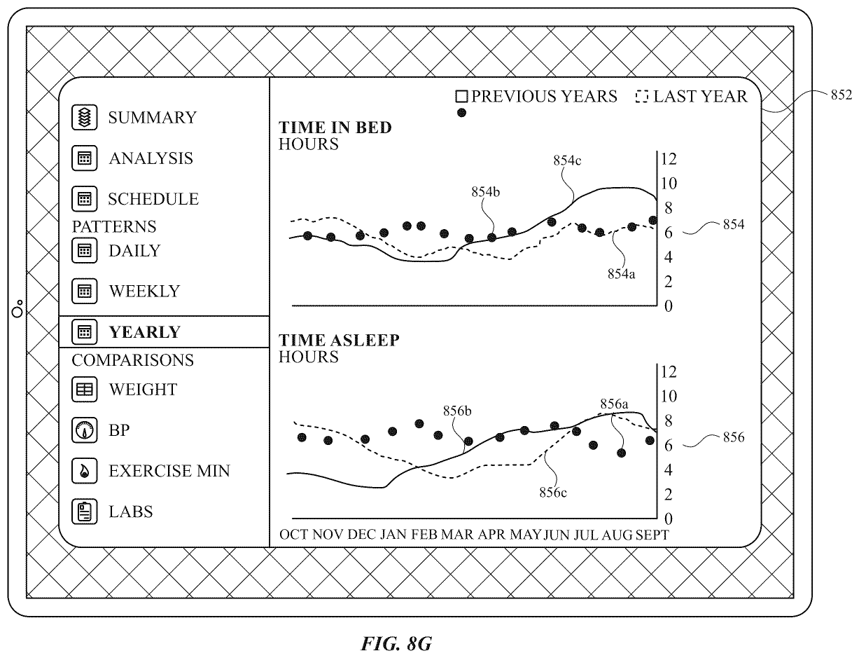

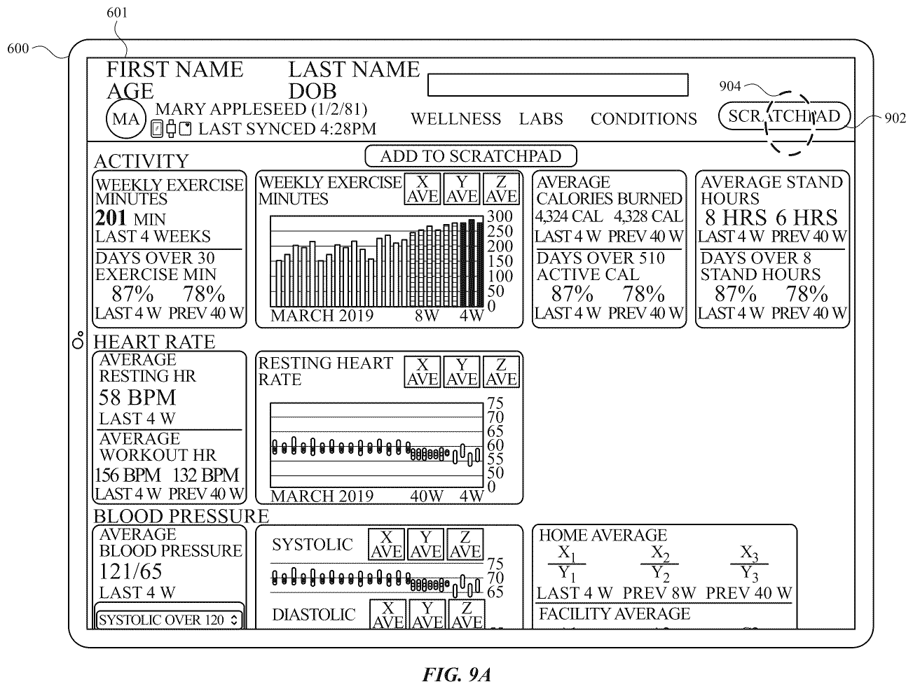

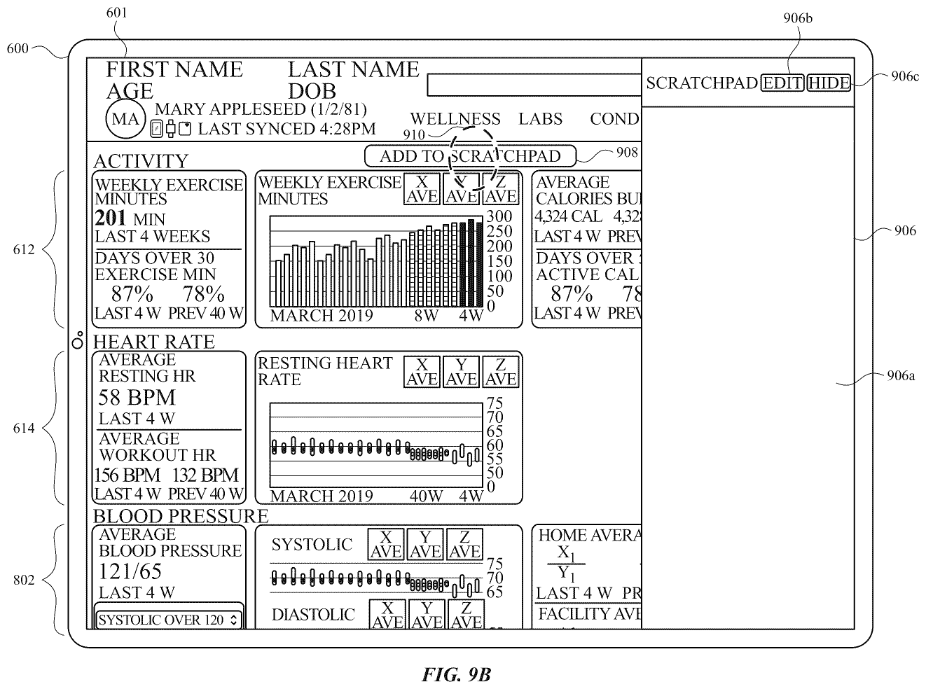

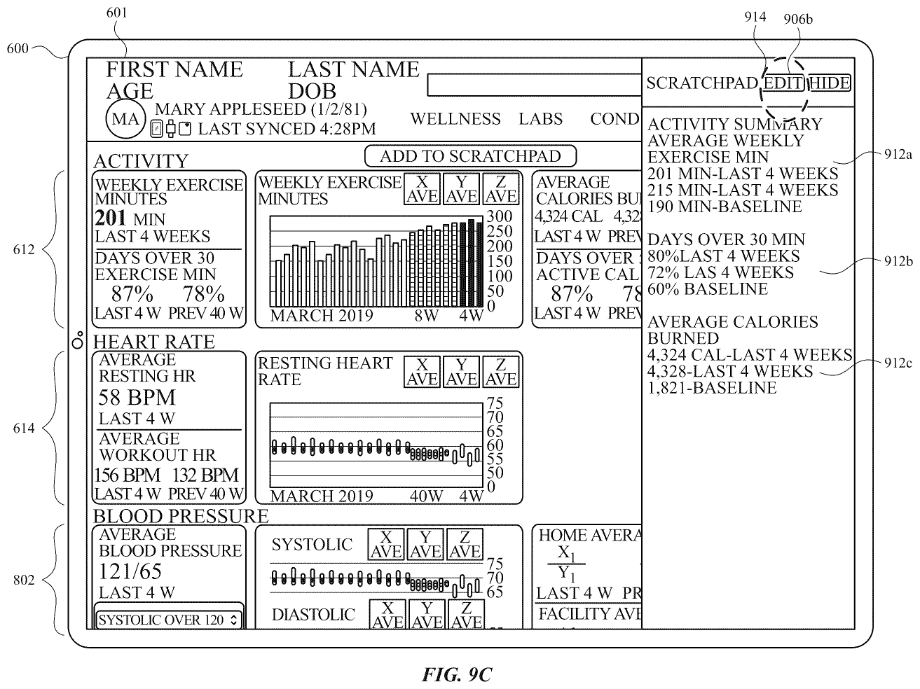

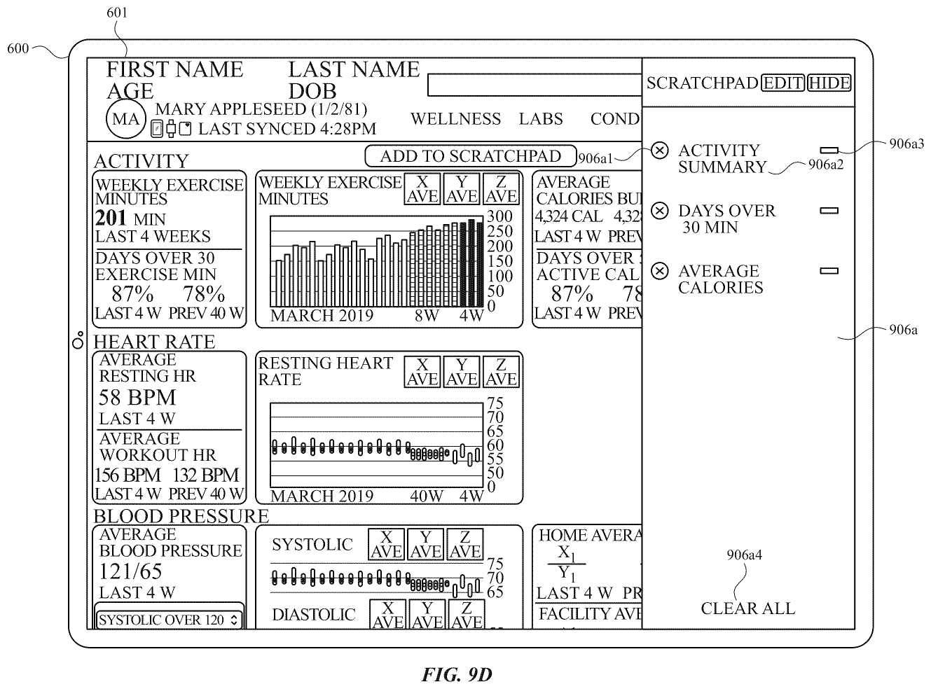

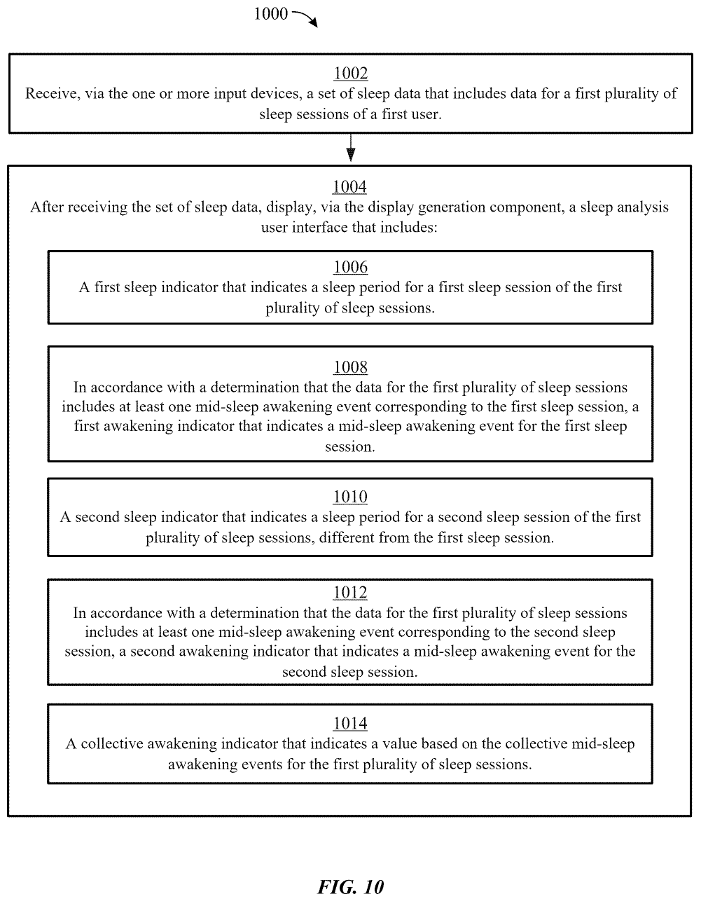

Below, FIGS. 1A-1B, 2, 3, 4A-4B, and 5A-5B provide a description of exemplary devices for performing the techniques for managing health data for a patient. FIGS. 6A-6K illustrate exemplary user interfaces for managing health data for a patient. FIGS. 7A and 7B are a flow diagram illustrating methods of managing health data for a patient in accordance with some embodiments. The user interfaces in FIGS. 6A-6K are used to illustrate the processes described below, including the processes in FIGS. 7A and 7B. FIGS. 8A-8G illustrate exemplary user interfaces for managing health data for a patient, specifically sleep-related data, in accordance with some embodiments. FIGS. 9A-9D illustrate exemplary user interfaces for managing health data for a patient, in accordance with some embodiments. FIG. 10 is a flow diagram illustrating a method for managing health data for a patient, specifically sleep-related data, using a computer system, in accordance with some embodiments. The user interfaces in FIGS. 8A-8G are used to illustrate the processes described below, including the processes in FIG. 10.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. These terms are only used to distinguish one element from another. For example, a first touch could be termed a second touch, and, similarly, a second touch could be termed a first touch, without departing from the scope of the various described embodiments. The first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage media), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

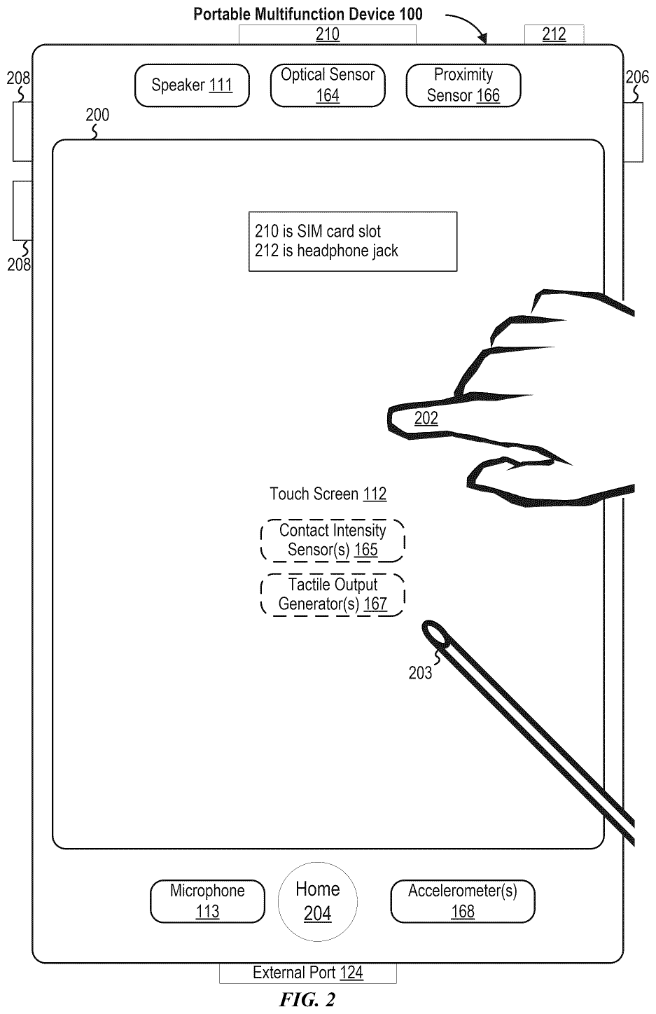

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures) as input. In some embodiments, the one or more input devices are integrated with the computer system. In some embodiments, the one or more input devices are separate from the computer system.

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), 6,570,557 (Westerman et al.), and/or 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

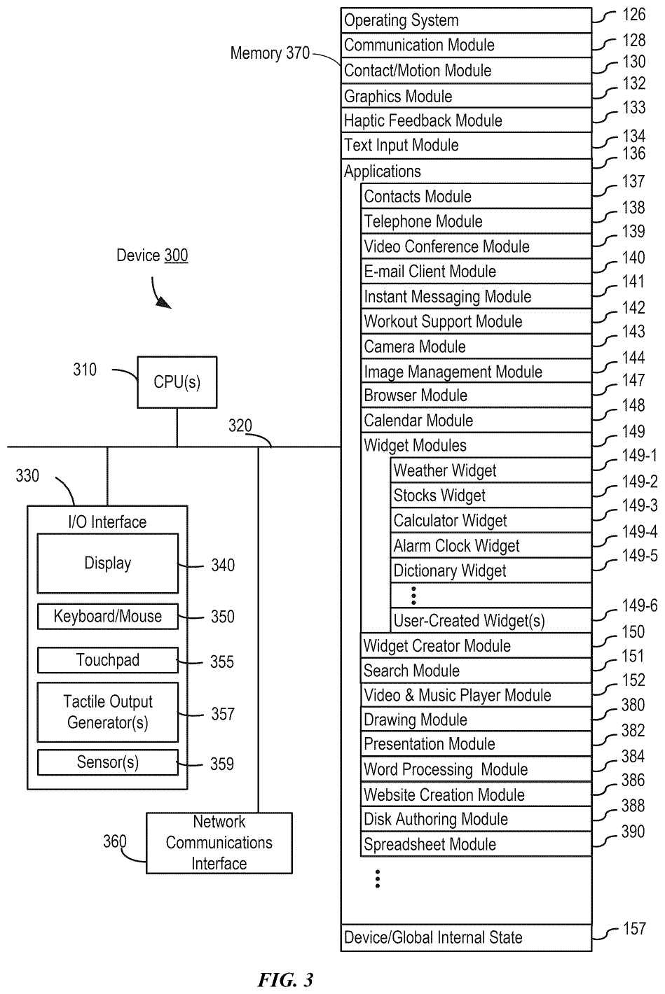

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE®, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript® file. In some embodiments, a widget includes an XML (Extensible Markup Language) file and a JavaScript® file (e.g., Yahoo!® Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

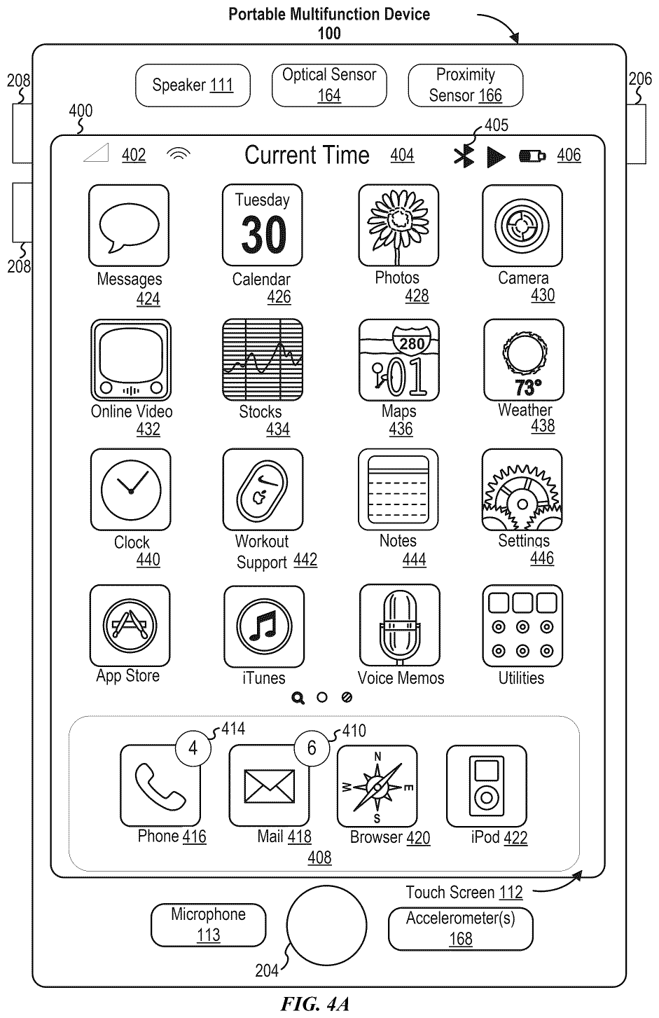

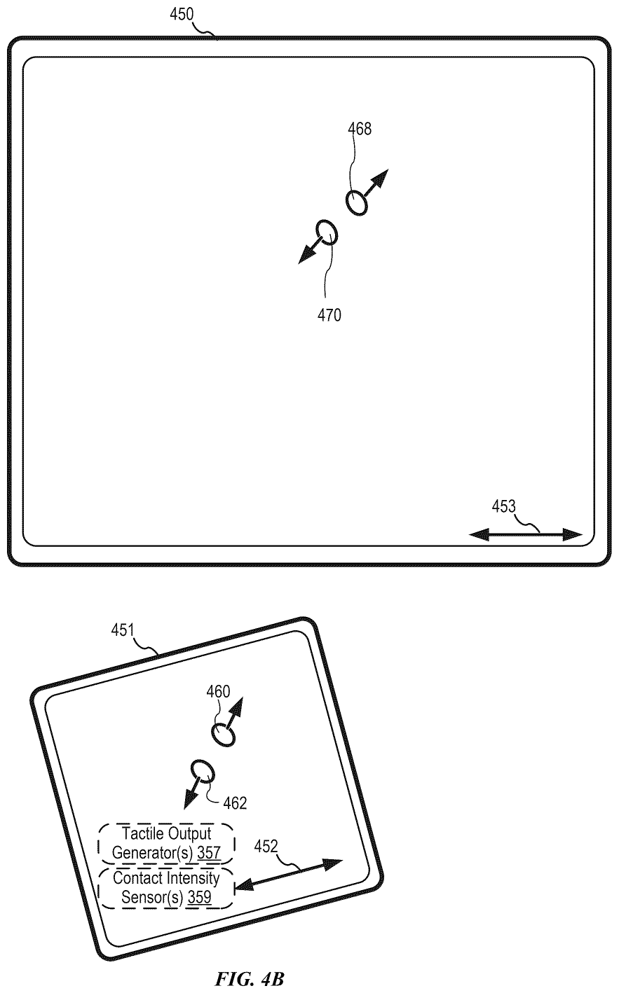

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch-sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contact 462 corresponds to 470). In this way, user inputs (e.g., contacts 460 and 462, and movements thereof) detected by the device on the touch-sensitive surface (e.g., touch-sensitive surface 451 in FIG. 4B) are used by the device to manipulate the user interface on the display (e.g., display 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

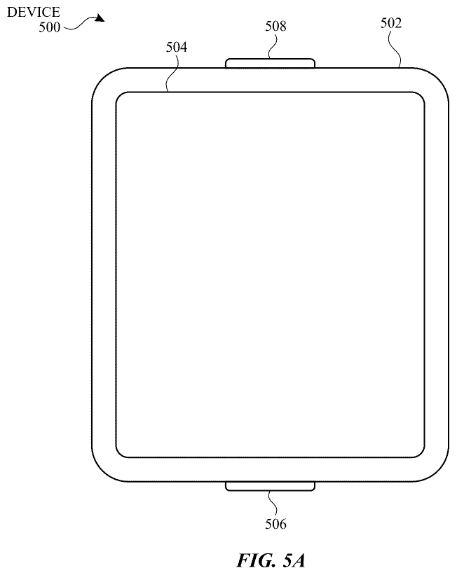

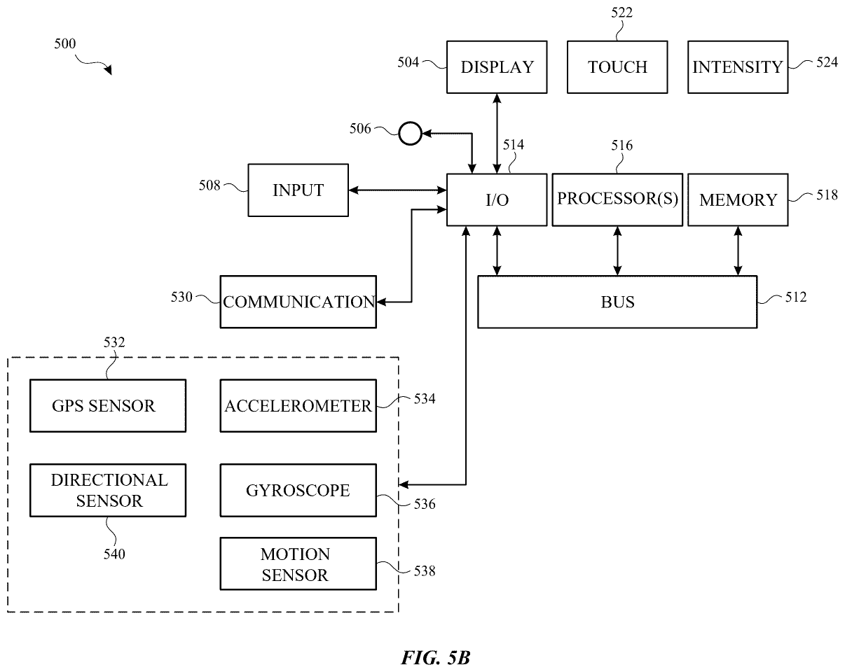

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage media, for storing computer-executable instructions, which, when executed by one or more computer processors 516, for example, can cause the computer processors to perform the techniques described below, including processes 700 and 1000 (FIGS. 7A-7B and 10). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray® technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6K illustrate exemplary user interfaces for managing health data for a patient, in accordance with some embodiments. The user interfaces in these figures are used to illustrate the processes described below, including the processes in FIGS. 7A and 7B.

FIGS. 6A-6K illustrate device 600 displaying user interfaces on display 601 (e.g., a display device or display generation component) for managing health data for a patient. In the embodiments depicted in the figures, the user interfaces on display 601 represent user interfaces that are displayed to a physician or medical provider, for example, for managing a patient's health data. In some embodiments, the user interfaces can be displayed on a different device, such as a laptop screen or a monitor, and the user interfaces can be interacted with via touch input or other input devices such as a cursor being controlled using a trackpad, mouse, or the like. In some embodiments, device 600 includes one or more features of devices 100, 300, or 500.

In FIG. 6A, device 600 depicts dashboard user interface 602, which represents a general overview of health data for a patient (Mary Appleseed). Dashboard UI 602 includes patient data 604, which includes the patient's name (e.g., MARY APPLESEED), date of birth (e.g., Jan. 2, 1981), a monogram (such as a visual representation corresponding to the patient, depicted in FIG. 6A as a circle with the letters “MA” inside of the circle), an indication of the most recent date/time that the patient's records were added or synced (e.g., LAST SYNCED 4:28 PM), and representations of different data sources (e.g., a smartwatch, a smartphone, health apps) that are used to measure/collect/record/provide the patient's health data that is being presented in dashboard UI 602. In some embodiments, the different data sources are typically associated with an account that is different from an account associated with device 600. For example, the different data sources are logged into a user account that is associated with the patient and separate from an account (e.g., a medical provider's account) that is associated with device 600. In some embodiments, the patient's user account is authorized (e.g., by the patient) to share the patient's health data with the account associated with device 600. In the embodiments discussed herein, some of the patient's health data is based on biometric measurements obtained from one or more of the different data sources. The biometric data can include heartrate information, electrocardiogram (ECG) measurements, weight measurements, blood pressure readings, and menstrual period data. The health data can include activity information such as the amount of time the patient exercises, the amount of calories burned by the patient, and the number of hours during which the patient is detected standing.

In some embodiments, patient data 604 is selectable to view additional patient details, which can be shown associated with patient data 604 (e.g., as a graphical element extending from, for example, the monogram) in response to an input (e.g., a tap, tap-and-hold, or hover gesture (e.g., using a cursor)) on patient data 604. Additional patient details can include health records provided from a hospital and a health clinic, and data indicating a time when the data was last synced from a respective source of the health data.

Dashboard UI 602 includes selectable tabs 606a-606c, which are selectable to view additional patient health records. For example, labs tab 606b is selectable to view and/or search detailed lab information for the patient, as discussed in greater detail below with respect to FIGS. 6I-6K.

Dashboard UI 602 includes patient overview banner 608, which provides an at-a-glance view of patient data, including representations of measurements for various health metrics such as the patient's Body Mass Index (BMI), exercise time, resting heartrate, blood pressure, and a number of health alerts (e.g., heart alerts such as high heartrate alerts, low heartrate alerts, irregular rhythm alerts). For some of the health metrics, the representations of measurements include indications of average measurements for the health metric for the past (e.g., prior year), which are shown in FIG. 6A as broken down into three bar graph representations that correspond to average measurements for the most recent four weeks (shown in a solid black bar), the average measurements for the eight weeks prior to the four-week measurements (shown in a hatched bar), and the average measurements for the forty weeks prior to the eight-week measurements (shown in a white bar). In some embodiments, the representations of the measurements in patient overview banner 608 can be selected to display additional details for the selected health metric, such as those discussed in greater detail below with respect to FIGS. 6B-6H. In some embodiments, selecting the representations of the measurements in patient overview banner 608 will scroll region 610 to the corresponding set of health metrics, as discussed below with respect to FIG. 6A.

Dashboard UI 602 also includes region 610, which includes sets of health metrics for different categories. For example, in FIG. 6A, region 610 includes activity metrics 612 and heart rate metrics 614. In some embodiments, additional health metrics, such as ECG metrics, weight metrics, blood pressure metrics, and menstrual cycle metrics, can be viewed by scrolling dashboard UI 602.

Each set of metrics includes tiles that contain data associated with the respective set of metrics, expanding on the summary of data for the respective metric represented in overview banner 608. For example, activity metrics 612 includes summary tile 612a, which includes an indication of the weekly average of exercise minutes recorded over the past four weeks, as well as an indication of the percentage of days over that four week period, and over a prior 40 week period, that the patient exercised for over a goal amount of time (30 minutes in this example). As shown in FIG. 6A, Mary exercised for more than 30 minutes a day 87% of the days in the past four weeks, and she exercised for more than 30 minutes a day 78% of the days in the prior 40 week period.

Activity metrics 612 also includes graph tile 612b, which represents a graph of the activity data. In FIG. 6A, graph tile 612b shows a graph of the weekly exercise minutes over the past year.

Activity metrics 612 includes calories tile 612c, which represents data indicating the average of calories Mary burned during the past four weeks, and during a prior 40 week period. Calories tile 612c shows that Mary averaged 4,324 calories burned a week during the past four week period, and she burned an average of 4,328 calories a week during the prior 40 week period. Calories tile 612c also indicates the percentage of days that Mary met a goal of actively burning 510 calories. For instance, Mary actively burned 510 calories 88% of the days in the four week period, and 74% of the days in the prior 40 week period.

Activity metrics 612 includes standing tile 612d, which represents data indicating the average number of hours that Mary was standing during the past four weeks, and during a prior 40 week period. Standing tile 612d shows that Mary averaged eight hours a day in which stood during the four week period, and she averaged six hours a day in the prior 40 week period. Standing tile 612d also indicates the percentage of days that Mary met her goal of standing for eight hours in a day. For instance, Mary stood for more than eight hours for 89% of the days in the four week period, and she stood for more than eight hours for 81% of the days in the prior 40 week period.

Heartrate metrics 614 include heartrate summary tile 614a, which includes an indication of Mary's average resting heartrate recorded over the past four weeks, as well as an indication of her average heartrate during workouts over that four week period, and over a prior 40 week period. As shown in FIG. 6A, Mary's average heartrate during workouts was 156 BPM over the past four weeks, and her average heartrate during workouts was 132 BPM during the prior 40 week period. Heartrate metrics 614 also include graph tile 614b, which shows a graph indicating Mary's heartrate measurements during the past year.

Each of the summary tiles in region 610 are selectable to view additional details for the health metric associated with the selected summary tile. For example, in response to detecting input 624 on activity summary tile 612a (shown in FIG. 6A), device 600 displays details UI 625 for the activity metrics, as shown in FIG. 6B. Details UI 625 includes additional details for the patient's health data that correspond to the selected health metric. For example, if heartrate summary tile 614a is selected, details UI 625 shows additional details for the patient's health data for the heartrate health metric.

Referring now to FIG. 6B, details UI 625 illustrates additional details of the activity metric. Various details of the activity metric can be viewed by selecting tabs 626. In FIG. 6B, summary tab 626-1 is selected (e.g., by default), and details UI 625 includes graphs 628-1, 628-2, and 628-3, each representing various aspects (e.g., measurements) of the patient's activity data. The data for each graph is displayed over a time variable (in this case, one year), which can be selected via time affordances 630, which include “all time” affordance 630-1, one year affordance 630-2, and four weeks affordance 630-3. In the example shown in FIG. 6B, one year affordance 630-2 is selected. Accordingly, each of the graphs represent activity data measurements over a one-year period (e.g., the most recent 52 weeks). Displaying each of the graphs over the same time variable (timeline) allows for comparing specific data points of the different graphs that correspond to a common time along the graph, as discussed in greater detail below.

Graph 628-1 represents measurements of Mary's daily active calories burned over the past year. The graph data is represented by bars 632, which are visually distinguished (e.g., using different variations of shading) to indicate a specific subset of the data. For example, bars 632-1 are shown in solid black and each represent a weekly average value of the daily active calories burned by Mary in a given week during the previous four week period. Similarly, bars 632-2 are shown in hatching and each represent a weekly average value of the daily active calories burned by Mary in a given week during the eight week period preceding the four week period. Finally, bars 632-3 are shown with no shading or hatching (e.g., solid white) and each represent a weekly average value of the daily active calories burned by Mary in a given week during the 40 week period preceding the eight week period. Details UI 625 also includes representations of an average value of the bars comprising a specific subset of the data in graph 628-1. For example, average 634-1 indicates that the average value of bars 632-1 is 1100, meaning that Mary averaged 1100 calories burned each day during the four week period. Similarly, average 634-2 indicates that the average value of bars 632-2 is 999, and average 634-3 indicates that the average value of bars 632-3 is 775.

Graph 628-2 represents measurements of Mary's daily exercise minutes over the past year—that is, the same previous year as that corresponding to the data in graph 628-1. The graph data is represented by bars 636, which are similar to bars 632. Bars 636-1 each represent a weekly average value of Mary's daily exercise minutes in a given week during the previous four week period. Bars 636-2 each represent a weekly average value of Mary's daily exercise minutes in a given week during eight week period preceding the four week period. Bars 636-3 each represent a weekly average value of Mary's daily exercise minutes in a given week during the 40 week period preceding the eight week period. Details UI 625 also includes representations of an average value of the bars comprising a specific subset of the data in graph 628-2. For example, average 638-1 indicates that the average value of bars 636-1 is 140, meaning that Mary averaged 140 exercise minutes each day during the four week period. Similarly, average 638-2 indicates that the average value of bars 636-2 is 125, and average 638-3 indicates that the average value of bars 636-3 is 110.

Graph 628-3 represents measurements of Mary's daily stand hours over the past year (the same previous year as that corresponding to the data in graph 628-1 and graph 628-2). The graph data is represented by bars 639, which are similar to bars 632 and 636. Bars 639-1 each represent a weekly average value of Mary's daily stand hours in a given week during the previous four week period. Bars 639-2 each represent a weekly average value of Mary's daily stand hours in a given week during eight week period preceding the four week period. Bars 639-3 each represent a weekly average value of Mary's daily stand hours in a given week during the 40 week period preceding the eight week period.

The data in each of the graphs is selectable to view additional details of the data represented by the graph that was selected, as well as additional details for data represented by the other graphs that were not directly selected and/or directly interacted with. For example, in FIG. 6C, device 600 detects input 640 (e.g., a tap gesture, tap-and-hold gesture, or hover gesture) on bar 632-3a of graph 628-1. In response, device 600 displays detail bubbles 641-1, 641-2, and 641-3 (sometimes referred to as “lollipops”), each corresponding to a data point of graphs 628-1, 628-2, and 628-3, respectively. Moreover, each detail bubble corresponds to the data point of its respective graph that has the same value along the timeline as the data point (bar 632-3a) that was selected in graph 628-1. For example, detail bubble 641-1 corresponds to bar 632-3a, detail bubble 641-2 corresponds to bar 636-3a, and detail bubble 641-3 corresponds to bar 639-3a, and bars 632-3a, 636-3a, and 639-3a each correspond to the same point (e.g., week 40) along the one-year timeline of graphs 628-1, 628-2, and 628-3, respectively.

As shown in FIG. 6C, detail bubbles 641-1, 641-2, and 641-3 are vertically aligned with each other, and are positioned outside of the respective graphs 628-1, 628-2, and 628-3. This allows a user to quickly associate each detail bubble with its corresponding graph, while also enabling simultaneous comparison of each set of data. In some embodiments, the detail bubbles are displayed vertically aligned and overlapping the graphs, as shown in FIG. 6D for example, such that the detail bubble is adjacent its corresponding bar. FIG. 6D shows detail bubbles 641-1, 641-2, and 641-3 displayed in response to input 642 on bar 632-3b and positioned adjacent corresponding bars 632-3b, 636-3b, and 639-3b, respectively. Bubbles 641-1, 641-2, and 641-3 are updated in FIG. 6D to reflect the values of the data represented by corresponding bars 632-3b, 636-3b, and 639-3b. This display may be generated to more closely associate the detail bubbles with the corresponding bars, while also allowing for simultaneous comparison of each set of data. Additionally, in some embodiments, the corresponding bars and/or regions adjacent the bars may be visually modified when one of the bars is selected. For example, the bars and/or adjacent regions may be highlighted, or the bars may be enlarged. Additionally, the detail bubbles may include a tail (e.g., tails 641-1c, 641-2c, and 641-3c in FIG. 6D), or other graphical indicator linking the respective detail bubble with the corresponding bar. The position of detail bubbles 641-1, 641-2, and 641-3 shown in FIG. 6D is an alternate position of the detail bubbles, where the detail bubbles are positioned adjacent and above the bars corresponding to the respective detail bubbles. Accordingly, the detail bubbles in FIGS. 6C and 6E-6H can also have a position similar to that shown in FIG. 6D. Similarly, the detail bubbles shown in FIG. 6D can alternatively have a position similar to that shown in FIG. 6C.

In the embodiment illustrated in FIGS. 6C and 6D, the detail bubbles include a cumulative total value of the activity data represented by its corresponding bar, as well as an indication of a portion of the time represented by the bar that the patient met a particular goal. For example, detail bubble 641-1 includes total value 641-la, which indicates, in FIG. 6C, that Mary actively burned a total of 8529 calories for the week represented by bar 632-3a (e.g., week 40). Similarly, detail bubble 641-1 shows, in FIG. 6D, that Mary actively burned a total of 9411 calories for the week represented by bar 632-3b (e.g., week 13). Detail bubble 641-1 also includes indication 641-1b showing that Mary met her goal of actively burning over 1000 calories in a day for six days of the week represented by bar 632-3a and for seven days of the week represented by bar 632-3b. Similarly, detail bubble 641-2 includes total value 641-2a indicating, in FIG. 6C, that Mary exercised for a total of 452 minutes during the week represented by bar 636-3a (e.g., week 40) and indicating, in FIG. 6D, that Mary exercised for a total of 473 minutes during the week represented by bar 636-3b (e.g., week 13). Detail bubble 641-2 also includes indication 641-2b showing that Mary met her goal of exercising for over 30 minutes a day for six days of the week represented by bar 636-3a and for seven days of the week represented by bar 636-3b. Detail bubble 641-3 includes total value 641-3a, indicating, in FIG. 6C, that Mary stood for at least a predetermined amount of time, per hour, for a total of 85 hours during the week represented by bar 639-3a (e.g., week 40) and indicating, in FIG. 6D, that Mary stood for at least a predetermined amount of time, per hour, for a total of 81 hours during the week represented by bar 639-3b (e.g., week 13). Detail bubble 641-3 also includes indication 641-3b showing that Mary met her goal of standing for at least a predetermined amount of time, per hour, for more than 12 hours in a day for six days of the week represented by bar 639-3a and for six days of the week represented by bar 639-3b.

As illustrated in FIGS. 6C and 6D, by selecting a bar for a particular week, a user can view details for different sets of data for the particular week (shown in the different detail bubbles), even though the data corresponds to different graphs. Moreover, the selection can be made in any of the graphs, and the corresponding detail bubbles are displayed for multiple graphs (e.g., all three graphs).

For example, in FIG. 6D, detail bubbles 641-1, 641-2, and 641-3 are updated in response to input 642 on bar 632-3b in graph 628-1, as discussed in greater detail above. In FIG. 6E, device 600 detects input 644 on bar 636-3a of graph 628-2, which corresponds to the same week (e.g., week 40) as bars 632-3a and 639-3a. Therefore, device 600 displays detail bubbles 641-1, 641-2, and 641-3 having the same details shown in FIG. 6C, even though the input was detected on a different bar of a different graph (bar 636-3a of graph 628-2, instead of bar 632-3a of graph 628-1). Accordingly, device 600 also displays detail bubbles 641-1, 641-2, and 641-3 having the same details as shown in FIGS. 6C and 6E, in response to detecting an input on bar 639-3a of graph 628-3.

Referring now to FIG. 6F, device 600 detects input 646 (e.g., a tap input) on four weeks affordance 630-3 and, in response, updates graphs 628-1, 628-2, and 628-3 based on the selected four-week time variable, as shown in FIG. 6G.

As shown in FIG. 6G, graphs 628-1, 628-2, and 628-3 are updated based on the selected four-week time variable, with each bar in each graph representing measured data for one day in the four week period. Accordingly, each bar 648 in graph 628-1 represents a measurement of daily active calories for a particular day during the most recent four week period. Similarly, each bar 650 in graph 628-2 represents daily exercise minutes for a particular day during the most recent four week period. Finally, each bar 652 in graph 628-3 represents daily stand hours for a particular day during the most recent four week period.

In FIG. 6G, device 600 detects input 654 on bar 650-1 of graph 628-2 and, in response, displays detail bubbles 656-1, 656-2, and 656-3 corresponding to the data represented by bars 648-1, 650-1, and 652-1, respectively. In the example shown in FIG. 6G, detail bubbles 656-1, 656-2, and 656-3 include the date of the corresponding data (e.g., Thursday, February 20^{th}), an indication of the measured data, an indication of the source of the measured data (e.g., a smartwatch), and an indication of whether the patient's goal was met for that particular day. For example, detail bubble 656-1 indicates that Mary burned 1100 calories on February 20^{th}, which met her goal of burning 1000 calories in a day, as indicated by checkmark 657. Watch icon 658 indicates that Mary's smartwatch measured the data. Similarly, detail bubble 656-2 indicates that Mary exercised for 150 minutes on February 20^{th}, which met her goal of exercising for at least 30 minutes that day (the data was measured using her smartwatch). Detail bubble 656-3 indicates that Mary's watch detected that she stood for 14 hours on February 20^{th}, which met her goal of standing for at least 12 hours.

FIG. 6H illustrates details UI 625, when “all time” affordance 630-1 is selected, and input 660 is detected on bar 662-1 of graph 628-1. In FIG. 6H, each bar in each graph represents measured data for one month of the total amount of time for which measurement data exists (e.g., three years in the current example). Accordingly, each bar 662 in graph 628-1 represents a measurement of daily active calories for a particular month. Similarly, each bar 664 in graph 628-2 represents daily exercise minutes for a particular month, and each bar 666 in graph 628-3 represents daily stand hours for a particular month.

In FIG. 6H, device 600 detects input 660 on bar 662-1 of graph 628-1 and, in response, displays detail bubbles 670-1, 670-2, and 670-3 corresponding to the data represented by bars 662-1, 664-1, and 666-1, respectively. In the example shown in FIG. 6H, detail bubbles 670-1, 670-2, and 670-3 include a weekly average value of the corresponding data, and an indication of the number of days for the month that the patient met their goal associated with the data. For example, detail bubble 670-1 indicates that Mary burned an average of 9201 calories per week for the month corresponding to bar 662-1, and that she met her goal of burning 1000 calories in a day for 28 of the 31 days in the corresponding month. Detail bubble 670-2 indicates that Mary exercised for an average of 770 minutes per week, and that she met her goal of exercising for at least 30 minutes that day for 28 of the 31 days in the month. Detail bubble 670-3 indicates that Mary stood for an average of 77 hours per week, and that she met her goal of standing at least a predetermined amount of time, per hour, for over 12 hours in a day for 28 of the 31 days in the month.

As illustrated in the figures and discussed above, the contents of the graphs and each detail bubble depend, in some instances, on the selected time variable for the graphs and the available data. For example, when the time variable is “one year,” each bar in the respective graphs represents a particular week of the previous 52 weeks, the data in the detail bubbles corresponds to a cumulative total value of the data represented by the corresponding bar, and the detail bubbles include an indication of whether various goals are met for the particular week (e.g., as shown in FIG. 6C). When the time variable is “all time,” each bar in the respective graphs represents a particular month, the data in the detail bubbles corresponds to a weekly average value of the data represented by the corresponding bar, and the detail bubbles include an indication of whether various goals are met for the particular month (e.g., as shown in FIG. 6H). When the time variable is “four weeks,” each bar in the respective graphs represents a particular day within the past four weeks, the data in the detail bubbles include an indication of a source of the measurement data, and the detail bubbles include an indication of whether various goals are met for the particular day (e.g., as shown in FIG. 6G). The values associated with the respective graphs and bars is representative of the available data and, therefore, these values can change based on the amount of available data and the measurements represented by the available data.

Referring now to FIG. 6I, device 600 displays labs UI 675, which is displayed in response to an input on labs tab 606b of FIG. 6A. Labs UI 675 includes representations of various test results from different laboratory tests. In some instances, the test results provide a visual indication (e.g., range 676, range 678, and range 680) of an acceptable range of test result values (e.g., measurements), and an indication (e.g., icon 682, icon 684, icon 686) of a measured test result value. As shown in FIG. 6I, the test result value (e.g., icon 686) can be displayed outside the acceptable range (e.g., range 680) to provide a visual indication of how much a particular test result value deviates from the acceptable range. In some embodiments, a representation of a test result value (e.g., icon 686) that is outside the acceptable range is visually distinguished from representations of other test result values, for example, by displaying the representation of the test result value in a different color.

The lab results can be filtered and sorted by selecting different affordances. For example, “All Labs” affordance can be selected to display (e.g., in alphabetical order) a list of all available measurements. Additionally, “Out of Range Labs” affordance can be selected to display only labs with measurements that are out of the acceptable range (e.g., including the date of out of range measurement and the range graph). In some embodiments, search box 687 can be used to search for individual lab results.

FIG. 6I also includes panel affordances 689, which can be selected to view various test panels. In some embodiments, the test panels associated with each of the panel affordances 689 are not necessarily test panels (e.g., singular, discrete test panels) existing in the patient's medical record, but rather, are panels that are created ad hoc by identifying various test results that are recorded in the patient's medical history, and using the test data to generate various panels that are typically built using the tests that were identified in the patient's medical data. In some embodiments, the panels are generated using a system or computer programs associated with labs UI 675 and operating, at least partially, at device 600. For example, the system receives the patient's medical records, identifies test results in the patient's medical records, identifies a set of panels that can be generated using the test results, generates the panels, and displays panel affordances 689, each affordance being associated with a different panel that was generated using the patient's medical data.

In FIG. 6J, labs UI 675 is replaced with metabolic panel UI 688 in response to selection of metabolic panel affordance 689-1. Metabolic panel UI 688 displays a metabolic panel that was generated as discussed above, where the panel includes a set of biometric measurements arranged in a table pattern, as shown in FIG. 6J. The table view in FIG. 6J can be scrolled to view all available measurements comprising the generated metabolic panel. Metabolic panel UI 688 further includes a set of pattern view affordances 690, which are selectable to rearrange the displayed pattern of the biometric measurements comprising the generated metabolic panel. In FIG. 6J, the table view affordance 690-1 is selected, and the biometric measurements are therefore arranged in the table view shown in FIG. 6J. Other patterns include bullet points, charts, and a fishbone diagram. Fishbone pattern affordance 690-2 is selectable to switch to a fishbone pattern arrangement of the biometric measurements comprising the generated metabolic panel, as shown in FIG. 6K.

In FIG. 6K, metabolic panel UI 688 is updated such that the biometric measurements of the generated metabolic panel are rearranged into a fishbone diagram pattern in response to selection of fishbone pattern affordance 690-2. Fishbone diagram 692 in FIG. 6K shows the same biometric data (e.g., data for the same particular time period) as the table in FIG. 6J, but in a fishbone diagram arrangement. In some embodiments, the fishbone pattern in FIG. 6K shows a predetermined number of predetermined types of measurements, such as those used to generate the metabolic panel, and therefore, cannot be scrolled to view all available measurements.

In FIG. 6K, metabolic panel UI 688 further includes representations 693 of other types of measurements that are not included in the fishbone diagram (e.g., measurements that are not used to generate the metabolic panel, but are otherwise available in the patient's medical data). In some embodiments, fishbone diagram 692 or any of representations 693 can be selected to display separate platters (e.g., pop-up UI elements) for each measurement, that includes a visual indication of the current measurement within a defined range. In some embodiments, platters for the measurements that are included in the fishbone diagram are shown before other platters or arranged having a preferential placement (e.g., first in an order of presentation).

In some embodiments, metabolic panel 688 in FIG. 6K can be scrolled to view different fishbone diagrams for different days of testing (e.g., in an order from most recent to oldest). In some embodiments, fishbone diagram 692 includes an indication of measurement types (e.g., “BUN,” “Sodium”) that are out of an acceptable range (e.g., acceptable measurement range). In some embodiments, when a measurement type in fishbone diagram 692 is selected (or hovered over), device 600 displays a detailed view (e.g., a pop-up) of data associated with the measurement type including, in some instances, changes in the measurement over time (e.g., in a chart view). In some embodiments, the detailed view of the data associated with the measurement type includes a list (or at least a partial list) of past measurements showing a date/time of each past measurement and a location (e.g., hospital or clinic) for each past measurement.

FIGS. 7A and 7B are a flow diagram illustrating a method for managing health data for a patient using an electronic device in accordance with some embodiments. Method 700 is performed at computer system (e.g., 100, 300, 500, 600) (e.g., a smartphone, a tablet, a laptop) that is in communication with a display generation component (e.g., 601) (e.g., a display controller, a touch-sensitive display system) and one or more input devices (e.g., a touch-sensitive surface). Some operations in method 700 are, optionally, combined, the orders of some operations are, optionally, changed, and some operations are, optionally, omitted.

In some embodiments, the electronic device (e.g., 600) is a computer system. The computer system is optionally in communication (e.g., wired communication, wireless communication) with a display generation component and with one or more input devices. The display generation component is configured to provide visual output, such as display via a CRT display, display via an LED display, or display via image projection. In some embodiments, the display generation component is integrated with the computer system. In some embodiments, the display generation component is separate from the computer system. The one or more input devices are configured to receive input, such as a touch-sensitive surface receiving user input. In some embodiments, the one or more input devices are integrated with the computer system. In some embodiments, the one or more input devices are separate from the computer system. Thus, the computer system can transmit, via a wired or wireless connection, data (e.g., image data or video data) to an integrated or external display generation component to visually produce the content (e.g., using a display device) and can receive, a wired or wireless connection, input from the one or more input devices.

As described below, method 700 provides an intuitive way for managing health data for a patient. The method reduces the cognitive burden on a user for managing health data for a patient, thereby creating a more efficient human-machine interface. For battery-operated computing devices, enabling a user to manage a patient's health data faster and more efficiently conserves power and increases the time between battery charges.

In method 700, the computer system (e.g., 600) displays (702), via the display generation component (e.g., 601), a plurality of graphical representations of data (e.g., 628-1, 628-2, 628-3) (e.g., charts (e.g., pie charts, radar charts) and/or graphs (e.g., line graphs, scatter plots, histograms)). The plurality of graphical representations of data include a first graphical representation of data (e.g., 628-1) corresponding to a first data set (e.g., 632) (e.g., data measuring daily active calories, weight data), and a second graphical representation of data (e.g., 628-2) corresponding to a second data set (e.g., 636) (e.g., measurement data that is different than the first measurement data; data measuring daily exercise minutes) that is different from the first data set. In some embodiments, the second graphical representation does not overlap the first graphical representation. In some embodiments, the second graphical representation is separate from the first graphical representation.

In method 700, the computer system (e.g., 600) detects (704), via the one or more input devices (e.g., 601), a first input (e.g., 640; 644; 654; 660) (e.g., a hover gesture (e.g., using a cursor); a tap-and-hold gesture) corresponding to the first graphical representation of data (e.g., 628-1). In some embodiments, the first input corresponds (e.g., is directed) to a location within the first graphical representation of data and outside of the second graphical representation of data.

In response to detecting the first input (e.g., 640; 644; 654; 660) corresponding to the first graphical representation of data, the computer system (e.g., 600) displays (706) a plurality of user interface objects (e.g., 641-1 to 641-3; 656-1 to 656-3; 670-1 to 670-3) (e.g., graphical representations, detail bubbles, lollipops, popup windows, and/or comment bubbles; a first plurality of user interface objects). The plurality of user interface objects include (708) a first user interface object (e.g., 641-1; 656-1; 670-1) associated with the first graphical representation of data (e.g., 628-1) and based on a first variable (e.g., a value along a first axis of the graphical representation (e.g., a specific week along a time axis) (e.g., week 40 as discussed with respect to FIG. 6C) (e.g., a specific day (e.g., February 20th) as discussed with respect to FIG. 6G) (e.g., a specific month as discussed with respect to FIG. 6H)) that is selected based on a location of the first input.

In accordance with a determination that the first input corresponds to a first location (e.g., a location along a first axis or two or more axes) in the first graphical representation of data (e.g., input 640 on bar 632-3a of graph 628-1), the first user interface object (e.g., 641-1) includes (710) a representation (e.g., 641-1a) (e.g., a graphical and/or textual representation) of a first subset of the first data set associated with (e.g., selected based on) the first variable (e.g., a first subset of data measuring daily active calories for the week associated with the user interface object). In some embodiments, the graphical representation of the data is a graph (e.g., bar graph) of data having an x-axis and a y-axis and the first user interface object includes a representation of the x and/or y values corresponding to the location of the first input.

In accordance with a determination that the first input corresponds to a second location in the first graphical representation of data (e.g., input 660 on bar 662-1 of graph 628-1) different from the first location (e.g., different along at least one axis), the first user interface object (e.g., 670-1) includes (712) a representation of a second subset of the first data set that is associated with the first variable (e.g., the contents of detail bubble 670-1) (e.g., a second subset of data measuring daily active calories for the week associated with the user interface object) and that is different than the first subset of the first data set (e.g., a subset that does not include or overlap with the first subset).

In response to detecting the first input (e.g., 640; 644; 654; 660) corresponding to the first graphical representation of data, the computer system (e.g., 600) displays the plurality of user interface objects (e.g., 641-1 to 641-3; 656-1 to 656-3; 670-1 to 670-3) (e.g., graphical representations, detail bubbles, lollipops, popup windows, and/or comment bubbles), including (714) a second user interface object (e.g., 641-2; 656-2; 670-2) associated with the second graphical representation of data (e.g., 628-2) and based on a second variable (e.g., the first variable (in some embodiments, the second variable is the same as the first variable)) (e.g., week 40 as discussed with respect to FIG. 6C) (e.g., a specific day (e.g., February 20th) as discussed with respect to FIG. 6G) (e.g., a specific month as discussed with respect to FIG. 6H) that is selected based on a location of the first input.

In accordance with a determination that the first input corresponds to the first location in the first graphical representation of data (e.g., input 640 on bar 632-3a of graph 628-1), the second user interface object (e.g., 641-2) includes (716) a representation (e.g., 641-2a) of a first subset of the second data set associated with the second variable (e.g., a first subset of data measuring daily exercise minutes for the week associated with the user interface object). Displaying the second user interface object including a representation of a first subset of the second data set associated with the second variable in accordance with a determination that the first input corresponds to the first location in the first graphical representation of data provides feedback to a user of the computer system that the first subset of the second data associated with the second variable corresponds to a value of the second variable that is based on the selected location in the first graphical representation of data (e.g., a value of the second variable that corresponds to a same relative location of the second graphical representation of data as the selected location in the first graphical representation of data). Displaying the second user interface object including the representation of the first subset of the second data set associated with the second variable in accordance with the determination that the first input corresponds to the first location in the first graphical representation of data also reduces the number of inputs that would be needed to display the corresponding second user interface object for the second graphical representation of data, by automatically displaying the second user interface object when the first input corresponds to the first location, thereby eliminating the need to make a subsequent selection on the second graphical representation of data. This also ensures that the second user interface object accurately corresponds to the selected location of the first graphical representation of data, by eliminating human error that could occur by inaccurately selecting a corresponding location on the second graphical representation of data. Providing improved feedback, reducing the number of inputs at the computer system, and performing an operation when a set of conditions has been met without requiring further input enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In accordance with a determination that the first input corresponds to the second location in the first graphical representation of data different from the first location (e.g., input 660 on bar 662-1 of graph 628-1), the second user interface object (e.g., 670-2) includes (718) a representation of a second subset of the second data set that is associated with the second variable (e.g., a second subset of data measuring daily exercise minutes for the week associated with the user interface object) and that is different than the first subset of the second data set (e.g., the contents of detail bubble 670-2). Displaying the second user interface object including a representation of a second subset of the second data set associated with the second variable in accordance with a determination that the first input corresponds to the second location in the first graphical representation of data provides feedback to a user of the computer system that the second subset of the second data associated with the second variable corresponds to a value of the second variable that is based on the selected location in the first graphical representation of data (e.g., a value of the second variable that corresponds to a same relative location of the second graphical representation of data as the selected location in the first graphical representation of data). Displaying the second user interface object including the representation of the second subset of the second data set associated with the second variable in accordance with the determination that the first input corresponds to the second location in the first graphical representation of data also reduces the number of inputs that would be needed to update display of the corresponding second user interface object for the second graphical representation of data, by automatically updating the second user interface object when the first input corresponds to the second location, thereby eliminating the need to make a subsequent selection on the second graphical representation of data. This also ensures that the second user interface object accurately corresponds to the selected location of the first graphical representation of data, by eliminating human error that could occur by inaccurately selecting a corresponding location on the second graphical representation of data. Providing improved feedback, reducing the number of inputs at the computer system, and performing an operation when a set of conditions has been met without requiring further input enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, displaying the plurality of user interface objects includes displaying the first user interface object (e.g., 641-1) positioned relative to (e.g., separate from (e.g., spaced apart from; outside the boundary of; and/or not overlapping with)) the first graphical representation of data (e.g., 628-1) (e.g., and the second graphical representation of data), and displaying the second user interface object (e.g., 641-2) positioned relative to (e.g., separate from (e.g., spaced apart from; outside the boundary of; and/or not overlapping with)) the second graphical representation of data (e.g., 628-2) (e.g., and the first graphical representation of data). Displaying the first user interface object positioned relative to the first graphical representation of data, and displaying the second user interface object positioned relative to the second graphical representation of data provides feedback to a user of the computer system that multiple representations of data are being presented and also allows for successive review of the first and second user interface objects with the first and second graphical representations of data. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, displaying the plurality of user interface objects includes displaying the first user interface object (e.g., 641-1) and the second user interface object (e.g., 641-2) having a same position (e.g., the first user interface object has a same position as the second user interface object) along an axis (e.g., a same position along an x-axis, and different positions along a y-axis). Displaying the first user interface object and the second user interface object having a same position along the axis provides feedback to a user of the computer system that the first user interface objects corresponds to a same relative location of the first graphical representation of data as the second user interface object does the second graphical representation of data, while also allowing for successive review of the first and second graphical representations. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently. In some embodiments, the first and second user interface objects are displayed having a vertical alignment. In some embodiments, the first graphical representation of data and the second graphical representation of data are aligned (e.g., an edge (e.g., right edge, left edge) of each graphical representation is aligned) with the axis (e.g., an axis of the display generation component; an x-axis or y-axis).

In some embodiments, the second variable is the first variable. In some embodiments, the variable (e.g., a value or position along a time axis (e.g. the week for the graphical representation of data along an axis of a year, divided into weeks) is common for the first and second graphical representations of data.

In some embodiments, the first variable and the second variable are time-based variables (e.g., the first variable is time, and the second variable is also time). In some embodiments, the time variable is a length of time measuring a specific amount of time (e.g., three years, one year, the current year, one month, four weeks). In some embodiments, the time variable is a total amount of time (“all time”) for which data is recorded/collected.

In some embodiments, the representation (e.g., 641-1a) of the first subset of the first data set (e.g., or the representation of the second subset of the first data set) includes a total value (e.g., a cumulative and/or summed value) of the first subset of the first data set (e.g., a total of daily active calories burned) determined (e.g., calculated) over a first subset of time (e.g., a week). In some embodiments, the representation (e.g., 641-2a) of the first subset of the second data set (e.g., or the representation of the second subset of the second data set) includes a total value of the first subset of the second data set (e.g., a total of daily exercise minutes) determined (e.g., calculated) over a second subset of time (e.g., the first subset of time; e.g., a week; a month). In some embodiments, the first subset of time is determined based on a current time range (e.g., four weeks; one year; all time) corresponding to the first data set (e.g., the amount of time over which data is represented for the first data set) (e.g., one year as indicated by selected one year affordance 630-2). In some embodiments, the second subset of time is determined based on a current time range (e.g., four weeks; one year; all time; the current time range corresponding to the first data set) corresponding to the second data set (e.g., the amount of time over which data is represented for the second data set) (e.g., one year as indicated by selected one year affordance 630-2). In some embodiments, the first/second subset of time is determined based on the current time range. For example, if the current time range is “all time,” the first/second subset of time is one month (e.g., 31 days). As another example, if the current time range is one year, the first/second subset of time is one week. As yet another example, if the current time range is four weeks, the first/second subset of time is one day.

In some embodiments, the representation of the first subset of the first data set (e.g., or the representation of the second subset of the first data set) includes an average value of the first subset of the first data set (e.g., an average of daily active calories burned) determined (e.g., calculated) over a third subset of time (e.g., a week; a month) (e.g., the contents of detail bubble 670-1 in FIG. 6H). In some embodiments, the representation of the first subset of the second data set (e.g., or the representation of the second subset of the second data set) includes an average value of the first subset of the second data set (e.g., an average of daily exercise minutes) determined (e.g., calculated) over a fourth subset of time (e.g., the third subset of time; e.g., a week; a month) (e.g., the contents of detail bubble 670-2 in FIG. 6H). In some embodiments, the third subset of time and the fourth subset of time are determined based on a current time range (e.g., four weeks; one year; all time) corresponding to the first data set and the second data set (e.g., the amount of time over which data is represented for the first data set and/or the second data set). In some embodiments, the first/second subset of time is determined based on the current time range. For example, if the current time range is “all time,” the first/second subset of time is one month (e.g., 28 days, 30 days, or 31 days). As another example, if the current time range is one year, the first/second subset of time is one week. As yet another example, if the current time range is four weeks, the first/second subset of time is one day.

In some embodiments, the computer system (e.g., 600) detects, via the one or more input devices (e.g., 601), a third input (e.g., 654) corresponding to the first graphical representation of data (e.g., or the second graphical representation of data (e.g., 628-2)) (e.g., the third input is detected while the current time range over which data is presented for the first data set and the second data set is a certain value (e.g., one week, two weeks, four weeks)). In response to detecting the third input, the computer system displays a first graphical indication of a source (e.g., 658) (e.g., a graphical representation of a smartwatch) associated with the first data set (e.g., displaying the first graphical indication of the source in lieu of the average value of the first subset of the first data set). Displaying the first graphical indication of a source associated with the first data set provides feedback to a user of the computer system that the first data set was collected using the source. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In response to detecting the third input, the computer system displays a second graphical indication of a source (e.g., a graphical representation of a smartwatch) associated with the second data set (e.g., watch icon in detail bubble 656-2 (similar to icon 658)) (e.g., displaying the second graphical indication of the source in lieu of the average value of the first subset of the second data set). Displaying the second graphical indication of a source associated with the second data set provides feedback to a user of the computer system that the second data set was collected using the source. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently. In some embodiments, the representation of the first subset of the first data set does not include an average value of the first subset of the first data set when the current time range over which data is presented for the first data set is a particular value such as, for example, four weeks. In some embodiments, the representation of the first subset of the first data set instead includes a representation of a device (e.g., a smartwatch) that is/was used for obtaining (e.g., measuring) the first data set. Similarly, in some embodiments, the representation of the first subset of the second data set instead includes a representation of the device that is/was used for obtaining the second data set when the current time range is the particular value (e.g., four weeks).

In some embodiments, the first data set (e.g., 632-3a) corresponds to a fifth subset of time (e.g., a week; a month), and the second data set (e.g., 636-3a) corresponds to a sixth subset of time (e.g., the fifth subset of time; a week; a month). In some embodiments, the representation of the first subset of the first data set (e.g., 641-1) (e.g., or the representation of the second subset of the first data set) includes an indication (e.g., 641-1b) (e.g., “6 days over 1000 CAL per day”; “28/31 days over 1000 CAL per day”) of a first portion of the fifth subset of time (e.g., a number of days in the week; a number of days in the month) during which a measured value of the first subset of the first data set (e.g., a measured value of daily active calories burned) exceeds a first predetermined threshold value (e.g., 900 calories; 1000 calories; 1200 calories). In some embodiments, the representation of the first subset of the second data set (e.g., 641-2) (e.g., or the representation of the second subset of the second data set) includes an indication (e.g., 641-2b) (e.g., “6 days over 30 min”; “28/31 days over 30 min”) of a first portion of the sixth subset of time (e.g., a number of days in the week; a number of days in the month) during which a measured value of the first subset of the second data set (e.g., a measured value of daily exercise minutes) exceeds a second predetermined threshold value (e.g., 15 minutes; 30 minutes; one hour).

In some embodiments, the first variable and the second variable have a first value (e.g., one year as indicated by the selected one year affordance 630-2) (e.g, a currently selected value for a range of time; e.g., one year, four weeks, all time). In some embodiments, the computer system (e.g., 600) detects, via the one or more input devices (e.g., 601), an input (e.g., 646) (e.g., a single input; a single input on a single selectable user interface object (e.g., a selection of a “current year” or a “current week” affordance)) corresponding to a request to adjust a value of the first variable and the second variable. In response to detecting the input corresponding to a request to adjust the value of the first variable and the second variable, and in accordance with a determination that the input corresponds to a first request to adjust the first variable and the second variable (e.g., selection of a value for a range of time; e.g., one year, four weeks, all time), the computer system changes the first value of the first variable and the second variable to a second value different from the first value (e.g., changing the first variable and the second variable from a value of one year to a value of four weeks). Changing the first value of the first variable and the second variable to a second value different from the first value in accordance with a determination that the input corresponds to a first request to adjust the first variable and the second variable reduces the number of inputs needed to set the value of the first and second variables to the second value by eliminating the need to individually select the second value for the first variable and for the second variable. Reducing the number of inputs needed to perform an operation enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In response to detecting the input corresponding to a request to adjust the value of the first variable and the second variable, and in accordance with a determination that the input corresponds to a second request to adjust the first variable and the second variable (e.g., selection of a value for a range of time; e.g., one year, four weeks, all time), changing the first value of the first variable and the second variable to a third value different from the first value and the second value (e.g., changing the first variable and the second variable from a value of four weeks (or one year) to a value of all time as shown in FIG. 6H). Changing the first value of the first variable and the second variable to a third value different from the first value and the second value in accordance with a determination that the input corresponds to a second request to adjust the first variable and the second variable reduces the number of inputs needed to set the value of the first and second variables to the third value by eliminating the need to individually select the third value for the first variable and for the second variable. Reducing the number of inputs needed to perform an operation enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, displaying the first user interface object (e.g., 641-1) includes replacing display of a portion of the first graphical representation of data (e.g., 628-1) with at least a portion of the first user interface object (e.g., the first user interface object is a pop-up that is overlaid on a portion of the first graphical representation of data). Replacing display of a portion of the first graphical representation of data with at least a portion of the first user interface object provides feedback to a user of the computer system that the displayed first user interface object corresponds to the first data set. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, displaying the second user interface object (e.g., 641-2) includes replacing display of a portion of the second graphical representation of data (e.g., 628-2) with at least a portion of the second user interface object (e.g., the second user interface object is a pop-up that is overlaid on a portion of the second graphical representation of data). Replacing display of a portion of the second graphical representation of data with at least a portion of the second user interface object provides feedback to a user of the computer system that the displayed second user interface object corresponds to the second data set. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, the first user interface object is displayed having an initial position relative to the first graphical representation of data (e.g., the position of detail bubble 641-1 in FIG. 6C) (e.g., an initial or first position adjacent or overlaying a portion of the first graph and determined based on the location of the first input on the first graph), and the second user interface object is displayed having an initial position relative to the second graphical representation of data (e.g., the position of detail bubble 641-2 in FIG. 6C) (e.g., an initial or first position adjacent or overlaying a portion of the second graph and determined based on the location of the first input on the first graph). In some embodiments, the computer system (e.g., 600) detects, via the one or more input devices (e.g., 601), a fourth input (e.g., 642) (e.g., a second input on the first graph) corresponding to the first graphical representation of data (e.g., 628-1). In response to detecting the fourth input corresponding to the first graphical representation of data, the computer system updates display of the plurality of user interface objects (e.g., based on the location of the fourth input on the first graphical representation of data). In some embodiments, updating display of the plurality of user interface objects includes displaying the first user interface object (e.g., 641-1) having an updated position relative to the first graphical representation of data (e.g., the position of detail bubble 641-1 in FIG. 6D) (e.g., the updated position is determined based on the first variable that is selected based on the location of the fourth input on the first graphical representation of data) and different from the initial position relative to the first graphical representation of data (e.g., ceasing display of the first user interface object at the initial position, and displaying the first user interface object at the updated position). In some embodiments, updating display of the plurality of user interface objects includes displaying the second user interface object (e.g., 641-2) having an updated position relative to the second graphical representation of data (e.g., the position of detail bubble 641-2 in FIG. 6D) (e.g., the updated position is determined based on the first variable that is selected based on the location of the fourth input on the first graphical representation of data) and different from the initial position relative to the second graphical representation of data (e.g., ceasing display of the second user interface object at the initial position, and displaying the second user interface object at the updated position).

Displaying the first user interface object having an updated position relative to the first graphical representation of data, and displaying the second user interface object having an updated position relative to the second graphical representation of data, provides feedback to a user that the selection of data associated with the first and second graphical representations of data has changed and also allows for successive review of the first and second user interface objects with the first and second graphical representations of data. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the computer system by enabling the user to use the system more quickly and efficiently.

In some embodiments, displaying the first user interface object having the updated position includes updating a representation of a subset of the first data associated with the first variable (e.g., see updated content in detail bubble 641-1 in FIG. 6D). In some embodiments, displaying the second user interface object having the updated position includes updating a representation of a subset of the second data associated with the first variable (e.g., see updated content in detail bubble 641-2 in FIG. 6D).

Note that details of the processes described above with respect to method 700 (e.g., FIGS. 7A and 7B) are also applicable in an analogous manner to other methods or corresponding user interfaces described herein. For example, method 1000 optionally includes one or more of the characteristics of the various methods described above with reference to method 700. For example, the interaction techniques of method 700 can be used to interact with sleep-related user interfaces of method 1000. For brevity, these details are not repeated.

FIGS. 8A-8G illustrate exemplary user interfaces for managing health data for a patient, specifically sleep-related data, in accordance with some embodiments. The user interfaces in these figures are used to illustrate the processes described below, including the processes in FIG. 10.

In FIG. 8A, device 600 is displaying dashboard user interface 602 on display 601. In comparison to the view of dashboard UI 602 seen in FIG. 6A, the UI has been scrolled (e.g., in response to a user input) to show different health metrics. Specifically, dashboard UI 602, in FIG. 8A, now includes blood pressure metrics 802 and sleep metrics 804. Blood pressure metrics 802 include blood pressure-related data in textual and graphical form. Similarly, sleep metrics 804 includes sleep-related data, including a summary tile 804a, a graph tile 804b, and goal tile 804c. Summary tile 804a shows both the average time in bed and the average sleep time for patient Mary, over the latest week (the week of Aug. 1-7, 2020). Graph tile 804b provides a graphical depiction of time in bed and time asleep over the most recent week (shown as a black bar), the 11 weeks before that (shown as gray bars), and the 40 weeks before those 11 weeks (shown as white bars). In some embodiments, graph tile 804b displays different periods of time (e.g., days, years), rather than weeks, or displays a period other than the last 52 weeks (e.g., the last week, the last 6 months, the last 2 years). Goal tile 804c provides data related to a sleep goal for Mary (e.g., a goal of 7 hours per night), as well as data on meeting that goal for the same periods as shown in graph tile 804b. In FIG. 8A, device 600 detects input 806 on summary tile 804a of sleep metrics 804.

In FIG. 8B, in response to input 806, device 600 displays detailed sleep summary interface 808, which includes a detailed summary of sleep data that includes time in bed graph 814 and time asleep graph 816. In FIG. 8B, because weekly time scale selector 810b is selected (e.g., as indicated by it being bolded), graphs 814 and 816 are displayed with a weekly scale. FIG. 8B also includes monthly time selector 810a and daily time selector 810c, which can be used to change the scales of the graphs to a monthly scale or a daily scale, respectively. Graphs 814 and 816 also include goal indicators 817a and 817b, respectively, that provide indications of whether the data for a given week met the target sleep goal (e.g., 7 hours per night). In FIG. 8B, sleep goal control 819 can be used to toggle on, or off, goal indicators 817a and 817b.

Detailed sleep summary interface 808 also includes tab selectors 812a-j. Summary tab indicator 812a is currently bolded, indicating that the summary tab is currently displayed. In the embodiment of FIGS. 8A-8G, elements that are shown as being visually emphasized via bolding can be visually emphasized in other ways (e.g., underlining, displaying with a different size or different color). Tab selectors 812b and 812d-f are discussed in more detail, below. Tab selectors 812g-j can be selected to provide comparison user interfaces of patient Mary's sleep data with weight data (tab selector 812g), blood pressure data (tab selector 812h), resting heart rate data (tab selector 812i), and glucose data (tab selector 812j) for patient Mary, respectively.

Graph 814 of FIG. 8B presents time in bed data for the most recent week (shown as a black bar), the 11 weeks before that (shown in hatched bars), and the 40 weeks before those 11 weeks (shown in white bars). In some embodiments, time in bed data is determined directly by device 600 (e.g., using a combination of a sleep schedule for patient Mary and absence of use of device 600 during bedtime periods in that schedule.) In some embodiments, time in bed is determined using one or more sensors of device 600 (e.g., accelerometers) that indicate that patient Mary was prone during bedtime periods in the sleep schedule. In some embodiments, time in bed data is determined by an external device that is in communication with device 600, such as a smart watch or a dedicated sleep monitoring device.

Graph 816 of FIG. 8B presents sleep data for the most recent week (shown as a black bar), the 11 weeks before that (shown in gray bars), and the 40 weeks before those 11 weeks (shown in white bars). In some embodiments, sleep data is determined directly using one or more sensors of device 600. In some embodiments, sleep data is determined by an external device that is in communication with device 600, such as a smart watch or a dedicated sleep monitoring device. In some embodiments, if a patient does not have a compatible external device for collecting sleep data, graph 816 is not displayed or is displayed with an absence of one or more data bars to indicate an absence of appropriate sleep data for a respective period of time.

In FIG. 8B, device 600 detects input 818 (e.g., a tap) on analysis tab selector 812b.

In FIG. 8C, in response to input 818, device 600 displays sleep analysis user interface 820. Sleep analysis user interface 820 includes a plurality of sleep data bars 822 that each present sleep data for a particular day. In FIG. 8C, data bars 822c-822i correspond to the last seven days of sleep data and data bars 822a and 822b correspond to the data for nine and eight days ago, respectively. Data bars 822c-822i are shown with lighter coloring to indicate that they are part of the most recent week while data bars 822a and 822b are displayed with a darker coloring to indicate that they are outside the most current week. In some embodiments, sleep analysis user interface 820 can be scrolled (e.g., via a swipe input) to show additional days of historical sleep data, for example, the remaining days of the 28 days (4 weeks) of data shown in displays sleep analysis user interface 820.

Data bar 822i, which corresponds to the previous night, August 7^{th}, includes base area 822il that is displayed in a first color that matches the color of indicator 824a of in bed column header 824. Base area 822il provides an indication of the time in bed for the previous night, August 7^{th}. Note that the plurality of data bars 822 are all aligned along time axis 828. Thus, in FIG. 8C, it can be seen that patient Mary was in bed from approximately 12 AM to just past 8 AM, which corresponds to the 6 hours and 15 minutes shown in the in bed data column with in bed column header 824. Data bar 822i also includes asleep insets 82212a-822i2c. The sleep insets are displayed in a color that matches the color of indicator 826a of asleep column header 826. Asleep insets 82212a-822i2c indicate periods during which patient Mary was asleep for the previous night, August 7^{th}. As seen in data bar 822i, Mary was asleep for three discrete periods (822i2a-822i2c) during the night of August 7^{th}, which total 5 hours, as shown in the asleep data column with asleep header 826. Thus, data bar 822i also indicates two periods of mid-sleep awakenings, 822i3a (between asleep insets 82212a and 822i2b) and 822i3b (between asleep insets 822i2b and 822i2c). In summary, data bar 822i provides an indication of the time in bed, time asleep, and mid-sleep awakenings, as well as the time of night at which each of those occured (e.g., with reference to axis 828). The remaining data bars provide similar information for their respective days. Note that for days in which asleep data is not available (e.g., because Mary was not wearing a smart watch and/or dedicated sleep tracker), such as August 5^{th}, corresponding to data bar 822g, no asleep insets are shown and a null value is shown for the asleep data column with asleep header 826.

In FIG. 8C, sleep analysis user interface 820 also includes awakenings control 830 that includes an indication of the average number of awakenings, per night, for the 28 days of sleep data shown in sleep analysis user interface 820. In some embodiments, days for which sleep data is not available (e.g., August 5^{th}) are excluded from the calculation of average number of awakenings, per night. In some embodiments, awakenings control 830 includes a different statistical value (e.g., a median, a mode) and/or shows the average for a period other than 28 days (e.g., the past week, the past month, the past year). Sleep analysis user interface 820 also includes a sleep schedule control 832 that includes an indication of the number of currently active sleep schedules or, in some embodiments, the number of sleep schedules applicable to the current period (e.g., the last 28 days) of sleep data. Sleep schedule control 832 is discussed in more detail in FIG. 8E.

In FIG. 8C, device 600 detects input 834 (e.g., a tap) on awakenings control 830.

In FIG. 8D, in response to input 834, device 600 modifies sleep analysis user interface 820, including plurality of data bars 822, to visually emphasize the mid-sleep awakening periods, include mid-sleep awakenings 822i3a and 822i3b. Device 600 also bolds awakenings control 830 to indicate that mid-sleep awakening periods are currently being emphasized. Thus, awakenings control 830 can be used to more easily identify mid-sleep awakenings periods in the larger set of sleep data shown in sleep analysis user interface 820. In FIG. 8D, device 600 detects input 836 (e.g., a tap) on sleep schedule control 832.

In FIG. 8E, in response to input 836, device 600 displays sleep schedule start indicators 838 and sleep schedule end indicators 840 in sleep analysis user interface 820. Device 600 also modifies the color of data bars 822 to emphasize the sleep schedule indicators. As noted in sleep schedule control 832, patient Mary has two currently active sleep schedules. As seen in FIG. 8E, Mary has a sleep schedule for weekdays (11 PM to 8 AM) and a different sleep schedule for weekends (12 AM to 9 AM). For example on the previous night, December 7^{th}, sleep schedule start indicator 838a corresponds to 11 PM and sleep schedule end indicator 840a corresponds to 8 AM. In contrast, sleep schedule start indicator 838b, for a Saturday, corresponds to 12 AM while sleep schedule end indicator 840b for that Saturday corresponds to 9 AM. Thus, sleep schedule control 832 can be used to display the sleep schedule in sleep analysis user interface 820. In some embodiments, awakenings control 830 and sleep schedule control 832 are mutually exclusive toggles such that toggling on one will toggle the other one off (if currently on) and vice versa. In FIG. 8E, device 600 detects input 842 (e.g., a tap) on tab selector 812e, which corresponds to a weekly patterns tab.

In FIG. 8F, in response to input 842, device 600 displays weekly pattern user interface 844. Weekly pattern user interface 844 includes time in bed graph 846 and time asleep graph 848. Time in bed graph 846 provides a comparison of time in bed data (in hours) for the previous 12 weeks (in white) as compared to the 40 weeks that preceded the previous 12 weeks (in gray), by day of the week (e.g., Monday to Sunday). Time in bed graph 846 provides a user with a visualization, by day of the week, of time in bed data for a more recent period (e.g., the previous 12 weeks or roughly ¼ of a year) compared to time in bed data for a longer, less recent period (e.g., the 40 weeks preceding the previous 12 weeks or roughly 3/4 of a year). Time in asleep graph 848 provides a comparison of time asleep data (in hours) for the previous 12 weeks (in white) as compared to the 40 weeks that preceded the previous 12 weeks (in black), by day of the week (e.g., Monday to Sunday). Time asleep graph 848 provides a user with a visualization, by day of the week, of asleep data for a more recent period (e.g., the previous 12 weeks or roughly ¼ of a year) compared to asleep data for a longer, less recent period (e.g., the 40 weeks preceding the previous 12 weeks or roughly 3/4 of a year). In FIG. 8F, device 600 detects input 850 on tab selector 812f, which corresponds to a yearly patterns tab.

In FIG. 8G, device 600, in response input 850, displays yearly pattern user interface 852 that includes time in bed graph 854 and time asleep graph 856. Time in bed graph 854 includes lines 854a, 854b, and 854c that correspond to the current year (e.g., the previous 12 months), the previous year before that, and the year prior to the previous year, respectively. Time in bed graph provides hourly time in bed data (across the y-axis) by time of year (across the x-axis). Time asleep graph 856 includes lines 856a, 856b, and 856c that correspond to the current year (e.g., the previous 12 months), the previous year before that, and the year prior to the previous year, respectively. Time asleep graph provides hourly asleep data (across the y-axis) by time of year (across the x-axis).

FIGS. 9A-9D illustrate exemplary user interfaces for managing, including copying, health data for a patient, in accordance with some embodiments.

In FIG. 9A, device 600 displays dashboard user interface 602a on display 601. Dashboard user interface 602a includes the features and functions of dashboard user interface 602 discussed above, with the addition of scratchpad control 902. In FIG. 9A, device 600 detects input 904 on scratchpad control 902.

In FIG. 9B, device 600 displays, in response to input 904, scratchpad region 906. Scratchpad region 906 includes scratchpad area 906a, edit affordance 906b, and hide affordance 906c. Scratchpad area 906, which is empty in FIG. 9B, can display data that has been entered into the scratchpad, as discussed below. Edit affordance 906b can be used modify data that has been entered into scratchpad area 906a, as discussed below. Hide affordance 906c, when selected, dismisses scratchpad region 906 (e.g., restoring user interface 602a on display 601 to the state shown in FIG. 9A). In FIG. 9B, device 600 displays, in response to input 904, add affordance 908. In some embodiments, device 600 displays a separate add affordance for each of the health metrics (e.g., health metrics 612, health metrics 614, health metrics 802) in dashboard user interface 602a. In some embodiments, add affordance 908 is associated with the currently top-most health metric (e.g., 612), such that scrolling dashboard user interface 602a to present a different health metric at the top would cause add affordance 908 to be associated with a different health metric. In FIG. 9B, device 600 detects input 910 (e.g., a tap) on add affordance 908.

In FIG. 9C, in response to input 910, device 600 adds (e.g., copies) to scratchpad area 906, selected data corresponding to health metrics 612 (e.g., the health metric that add affordance 906 is currently associated with), including set of summary data 912a, set of greater-than-goal data 912b, and set of calorie data 912c. In some embodiments, the data includes from multiple different periods of time (e.g., last 4 weeks, last 12 weeks, and/or last 40 weeks). In some embodiments, selection of an add affordance that corresponds to a different health metric (e.g., health metrics 614 or health metrics 802) would add selected data corresponding to that respective health metric to be added to scratchpad area 906. In FIG. 9C, device 600 detects input 914 on edit affordance 906b. In some embodiments, selection of multiple add affordances would cause data to be added, in sequence (e.g., later data would be appended to existing data), to the scratchpad. Thus, a user can add any amount of desired data to the scratchpad, without adding undesired data.

In FIG. 9D, in response to input 914, displays modified scratchpad area 906a that includes a plurality of categories of data that has been added to the scratchpad. The plurality of categories include category 906a2 (“Activity Summary”), which is displayed in conjunction with remove affordance 906a and expand affordance 906c. Remove affordance 906a1, when selected, removes category 906a2 from the scratchpad, without removing the remaining categories (e.g., “Days Over 30M”). Expand affordance 906a3, when selected, expands category 906a2 to show the full set of data for Activity Summary, as seen in FIG. 9C. The remaining categories in modified scratchpad area 906a each include their own remove and expand affordances, with similar functionality. Modified scratchpad area 906a also includes clear all affordance 906a4 that, when selected, causes all data to be cleared from the scratchpad. In some embodiments, a user can copy the data in scratchpad area 906 and/or modified scratchpad area 906a to a system clipboard for exporting to another application. In some embodiments, the data in scratchpad area 906 and/or modified scratchpad area 906a can be exported to a communication application (e.g., a messaging application, an email application) shared with another device and/or user.

FIG. 10 is a flow diagram illustrating a method for managing sleep-related data for a patient using a computer system in accordance with some embodiments. Method 1000 is performed at a computer system (e.g., a tablet computer, a personal computer, a smart phone, 100, 300, 500) that is in communication with a display generation component (e.g., an integrated display, an external display, a display monitor, a display adapter) and one or more input devices (e.g., a touch-sensitive surface, a mouse, a keyboard, a data transmission bus). Some operations in method 1000 are, optionally, combined, the orders of some operations are, optionally, changed, and some operations are, optionally, omitted.

As described below, method 1000 provides an intuitive way for managing sleep-related data for a patient. The method reduces the cognitive burden on a user for managing sleep-related data for a patient, thereby creating a more efficient human-machine interface. For battery-operated computing devices, enabling a user to manage sleep-related data for a patient faster and more efficiently conserves power and increases the time between battery charges.

The computer system receives (1002), via the one or more input devices (e.g., 601), a set of sleep data that includes data for a first plurality of sleep sessions (e.g., data corresponding to 822a-822i) (e.g., a night of sleep; a period of time between when a sleep session start criteria is met (e.g., when sleep is first detected after a period of non-sleep; when sleep is first detected within a predetermined period of time (e.g., a set bed time) and when a sleep session end criteria is met (e.g., a period of wakefulness that lasts more than a predetermined period of time; a period of wakefulness that is detected outside of the predetermined period of time)) of a first user (e.g., patient Mary Appleseed of FIG. 8A). In some embodiments, the sleep data is collected via one or more sensors (e.g., heart rate monitors, pressure sensors, and/or motion sensors) of the computer system. In some embodiments, the sleep data is collected by an external device (e.g., a smart watch; a dedicated sleep tracking device) and transmitted to, or retrieved by, the computer system. In some embodiments, the plurality of sleep sessions are the combined sleep sessions for a predetermined period of time (e.g., a week, a month, a year).

After receiving the set of sleep data, the computer system displays (1004), via the display generation component (e.g., 601), a sleep analysis user interface (e.g., 820) that includes (e.g., concurrently includes) a first sleep indicator (e.g., 82212a, 822i2b, 822i2c) (e.g., a graphical object) that indicates (1006) a sleep period (e.g., a period during which the data indicates that the first user is asleep) for a first sleep session (e.g., 822i, August 7th) (e.g., a first night of sleep) of the first plurality of sleep sessions. In some embodiments, the indicator indicates a time and duration of the sleep period. In some embodiments, multiple sleep indicators are displayed for the first sleep session.

The sleep analysis user interface further includes (e.g., concurrently includes), in accordance with a determination that the data for a first plurality of sleep sessions includes at least one mid-sleep awakening event corresponding to the first sleep session, a first awakening indicator (e.g., 822i3a, 822i3b) (e.g., a graphical object) that indicates (1008) a mid-sleep awakening event (a period of time during which the data indicates that the first user has awakened after falling asleep during the sleep session; a non-terminal awakening event during the sleep session; a discrete mid-sleep awakening event) for the first sleep session (in some embodiments, that is independent of awakening events, if any, of the second sleep period). In some embodiments, the indicator indicates a time and duration of the mid-sleep awakening event. In some embodiments, multiple awakening indicators are displayed for the first sleep session.

The sleep analysis user interface further includes (e.g., concurrently includes), a second sleep indicator (e.g., asleep periods for 822b) (e.g., a graphical object) that indicates (1010) a sleep period (e.g., a period during which the data indicates that the first user is asleep) for a second sleep session of the first plurality of sleep sessions, different from the first sleep session (e.g., a second night of sleep). In some embodiments, the indicator indicates a time and duration of the sleep period. In some embodiments, multiple sleep indicators are displayed for the second sleep session.

The sleep analysis user interface further includes (e.g., concurrently includes), in accordance with a determination that the data for a first plurality of sleep sessions includes at least one mid-sleep awakening event corresponding to the second sleep session, a second awakening indicator (e.g., mid-sleep awakening periods for 822b) (e.g., a graphical object) that indicates (1012) a mid-sleep awakening event (e.g., a period of time during which the data indicates that the first user has awakened after falling asleep during the sleep session; a non-terminal awakening event during the sleep session) for the second sleep session (in some embodiments, that is independent of awakening events, if any, of the first sleep period). In some embodiments, the indicator indicates a time and duration of the mid-sleep awakening event. In some embodiments, multiple awakening indicators are displayed for the second sleep session.

The sleep analysis user interface further includes (e.g., concurrently includes), a collective awakening indicator (e.g., 830) that indicates (1014) a value (e.g., (1.5 per night” in 830) (e.g., a numerical value (e.g., an average, a cumulative value, a median, a mode, a delta from a previous plurality of sleep sessions; a derived value; a value based on a plurality of mid-sleep awakening events)) based on the collective mid-sleep awakening events for the first plurality of sleep sessions (e.g., including the first session and the second session). Conditionally displaying a first and/or second awakening indicator based on the received sleep data provides the user with visual feedback about the state of the system, specifically the data received by the system, which provides improved visual feedback.

In some embodiments, the value based on the collective mid-sleep awakening events for the first plurality of sleep sessions is an average of mid-sleep awakening events for the first plurality of sleep sessions (e.g., 830) (e.g., the total of mid-sleep awakenings events divided by the number of sleep sessions in the plurality of sleep sessions). Displaying an average number of mid-sleep awakening events for the first plurality of sleep sessions provides the user with visual feedback about the state of the system, specifically the mid-sleep awakening data received by the system, which provides improved visual feedback.

In some embodiments, the sleep analysis user interface includes (e.g., concurrently includes) a first time in bed indicator (e.g., 822i1) (e.g., a graphical indicator; an alphanumeric value (e.g., “8 hours and 10 minutes”)) that indicates a total amount of time that the first user was in bed for the first sleep session and a second time in bed indicator (e.g., in bed portion of 822b) that indicates a total amount of time that the first user was in bed for the second sleep session. In some embodiments, the time in bed is determined based on data from one or more sensors (e.g., orientation sensors). Displaying time in bed indicators provides the user with visual feedback about the state of the system, specifically the data received by the system, which provides improved visual feedback.

In some embodiments, the sleep analysis user interface includes a first user-interactive graphical user interface object (e.g., 830) (e.g., an awakenings affordance). In some embodiments, the computer system receives, via the one or more input devices, a first input (e.g., a tap input, a mouse click, a key press) corresponding to the first user-interactive graphical user interface object. In response to receiving the first input, the computer system modifies a visual appearance of the first sleep indicator and/or the visual appearance of the first awakening indicator to emphasize the visual appearance of the first awakening indicator (e.g., appearance of 822i in FIG. 8D) (e.g., relative to the visual appearance of the first sleep indicator). In some embodiments, in response to receiving the first input, modifying a visual appearance of the second sleep indicator and/or the visual appearance of the second awakening indicator to emphasize the visual appearance o the second sleep indicator. Modifying a visual appearance of the first sleep indicator and/or the visual appearance of the first awakening indicator to emphasize the visual appearance of the first awakening indicator provides the user with an emphasized visualization option for viewing the mid-sleep awakening data, which provides improved visual feedback.

In some embodiments, after receiving the set of sleep data, the computer system displays, via the display generation component, a sleep summary user interface (e.g., 808) that includes (e.g., concurrently includes) a first sleep value indicator (e.g., 12 week portion of 814) that indicates a value of a first sleep parameter (e.g., a sleep-related metric (e.g., time in bed; time asleep)) for the first plurality of sleep sessions and a second sleep value indicator (e.g., 40 week portion of 814) that indicates a value of the first sleep parameter (e.g., a sleep-related metric (e.g., time in bed; time asleep)) for a second plurality of sleep sessions, that is different than the first plurality of sleep sessions. In some embodiments, the first and second plurality of sleep sessions are non-overlapping, consecutive periods of time. In some embodiments, the first and second plurality of sleep sessions are overlapping (e.g., the current/immediate last 12 weeks and the previous 40 weeks). In some embodiments, the first and second plurality of sleep sessions include differing numbers of sleep sessions (e.g., 12 weeks' worth of sessions and 40 weeks' worth of sessions). Displaying indicators for the same first sleep parameter for both the first and second plurality of sleep sessions provides the user with visual feedback as the data pertaining to both the data for the first and second plurality of sleep sessions, which provides improved visual feedback.

In some embodiments, the set of sleep data that includes data for the first plurality of sleep sessions of the first user includes data collected by an external electronic device (e.g., a dedicated sleep tracking device having sensors and software for collecting sleep related data) having one or more sensors configured to collect sleep data.

In some embodiments, the set of sleep data that includes data for the first plurality of sleep sessions of the first user includes data corresponding to a third sleep session (e.g., 822c, August 1st) that does not include data collected by an external electronic device (e.g., a dedicated sleep tracking device having sensors and software for collecting sleep related data) having one or more sensors configured to collect sleep data (e.g., the data for the third sleep session includes only data collected directly by the computer system and/or data that is manually entered by a user). In some embodiments, the sleep analysis user interface does not include an indicator of a mid-sleep awakening event for the third sleep session (e.g., 822c does not include mid-sleep awakenings) (e.g., any indicators of mid-sleep awakening events for the third sleep session). In some embodiments, the sleep analysis user interface does not include an indication of a total sleep time for the third sleep session. In some embodiments, the sleep analysis user interface does include an indication of a total time in bed for the third sleep session. Excluding, from the sleep analysis user interface, indicators for mid-sleep awakening events for the third sleep session, when the data for that session does not include data from an external electronic having one or more sensors configured to collect sleep data, provides the user with an indication as to the nature/characteristics/source of that data, which provides improved visual feedback. Doing so also automatically excludes such indicators when the data meets the condition of not including the requisite type of data, which performs an operation when a set of conditions has been met without requiring further user input.

In some embodiments, prior to displaying the sleep analysis user interface, the computer system displays a health summary user interface (e.g., 602) (an interface that summarizes parameters for a plurality of health-related topics) that includes (e.g., concurrently includes) a sleep user-interactive graphical user interface object (e.g., 804) (e.g., a summary of certain sleep-related data) that includes an indication of a value of a second sleep parameter (e.g., a sleep-related metric (e.g., time in bed; time asleep)). In some embodiments, the second sleep parameter is the same as the first sleep parameter. The health summary user interface further includes (e.g., concurrently includes) a first health category user-interactive graphical user interface object that includes an indication of a value of a first health parameter (e.g., 802) (e.g., blood pressure; menstruation, exercise/physical activity; weight; heart rate), wherein the first health parameter is not associated with a sleep parameter. The health summary user interface further includes (e.g., concurrently includes) a second health category user-interactive graphical user interface object (e.g., 612) that includes an indication of a value of a second health parameter (e.g., blood pressure; menstruation, exercise/physical activity; weight; heart rate), wherein the second health parameter is not associated with a sleep parameter. While displaying the health summary user interface, the computer system receives, via the one or more input devices, a first set of one or more inputs that includes a second input (e.g., 806) (e.g., a tap input, a mouse click, a key press) corresponding to the sleep user-interactive graphical user interface object. In response to receiving the first set of one or more inputs, the computer system displays the sleep analysis user interface (e.g., 820). Displaying a sleep user-interactive graphical user interface object along with other health category user-interactive graphical user interface objects provides the user with feedback as to the different types of health-related data available on the computer system, which provides improved visual feedback.

In some embodiments, the sleep analysis user interface includes a second user-interactive graphical user interface object (e.g., 832) (e.g., a sleep schedule affordance). In some embodiments, the computer system receives, via the one or more input devices, a third input (e.g., 836) (e.g., a tap input, a mouse click, a key press) corresponding to the second user-interactive graphical user interface object. In response to receiving the third input, the computer system displays, in the sleep analysis user interface, an indication (e.g., 838, 840) (e.g., a set of lines indicate a period of time (e.g., 10 PM to 7 AM)) of a pre-selected (e.g., via previous user input/user selection) sleep schedule corresponding to the first sleep session. In some embodiments, in response to receiving the third input, the computer system displays, in the sleep analysis user interface, an indication of a pre-selected (e.g., via previous user input/user selection) sleep schedule corresponding to the second sleep session. In some embodiments, the indication of a pre-selected sleep schedule corresponding to the first sleep session provides a visual indication of a relationship between the sleep period for the first sleep session and a pre-schedule period for the first sleep session. Displaying, in the sleep analysis user interface, an indication of a pre-selected sleep schedule corresponding to the first sleep session provides the user with information as to what sleep schedules were previously selected, which provides improved visual feedback.

Note that details of the processes described above with respect to method 1000 (e.g., FIG. 10) are also applicable in an analogous manner to the methods described herein. For example, method 700 optionally includes one or more of the characteristics of the various methods described above with reference to method 1000. For example, sleep-related user interfaces of method 1000 can be interacted with according to the techniques of method 700. For brevity, these details are not repeated above.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve management of a patient's health data. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater relevance to the patient's health. Accordingly, use of such personal information data enables users to have calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of healthcare services, the present technology can be configured to allow users to select to “opt in” or “opt out” of participation in the collection of personal information data during registration for services or anytime thereafter. In another example, users can select not to provide health data for targeted content delivery services. In yet another example, users can select to limit the length of time health data is maintained or entirely prohibit the development of a health profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the medical providers, or publicly available information.

What is claimed is:

1. A computer system that is configured to communicate with a display generation component and one or more input devices, the computer system comprising:
one or more processors; and
memory storing one or more programs configured to be executed by the one or more processors, the one or more programs including instructions for:
displaying, via the display generation component, a plurality of graphical representations of data, including:
a first graphical representation of data corresponding to a first data set; and
a second graphical representation of data corresponding to a second data set that is different from the first data set;
detecting, via the one or more input devices, a first input corresponding to the first graphical representation of data; and
in response to detecting the first input corresponding to the first graphical representation of data, displaying a plurality of user interface objects, including:
a first user interface object associated with the first graphical representation of data and based on a first variable that is selected based on a location of the first input, wherein displaying the first user interface object includes replacing display of a portion of the first graphical representation of data with at least a portion of the first user interface object, and wherein the first user interface object includes:
in accordance with a determination that the first input corresponds to a first location in the first graphical representation of data, a representation of a first subset of the first data set associated with the first variable; and
in accordance with a determination that the first input corresponds to a second location in the first graphical representation of data different from the first location, a representation of a second subset of the first data set that is associated with the first variable and that is different than the first subset of the first data set; and
a second user interface object associated with the second graphical representation of data and based on a second variable that is selected based on a location of the first input, wherein displaying the second user interface object includes replacing display of a portion of the second graphical representation of data with at least a portion of the second user interface object, and wherein the second user interface object includes:
in accordance with a determination that the first input corresponds to the first location in the first graphical representation of data, a representation of a first subset of the second data set associated with the second variable; and
in accordance with a determination that the first input corresponds to the second location in the first graphical representation of data different from the first location, a representation of a second subset of the second data set that is associated with the second variable and that is different than the first subset of the second data set.

2. The computer system of claim 1, the one or more programs further including instructions for:
detecting, via the one or more input devices, a second input, wherein the second input corresponds to the second graphical representation of data; and
in response to detecting the second input, displaying a second plurality of user interface objects, including:
a third user interface object associated with the first graphical representation of data and based on a third variable that is selected based on a location of the second input, including:
in accordance with a determination that the second input corresponds to a first location in the second graphical representation of data, a representation of a third subset of the first data set associated with the third variable; and
in accordance with a determination that the second input corresponds to a second location in the second graphical representation of data different from the first location, a representation of a fourth subset of the first data set that is associated with the third variable and that is different than the third subset of the first data set; and
a fourth user interface object associated with the second graphical representation of data and based on a fourth variable that is selected based on a location of the second input, including:
in accordance with a determination that the second input corresponds to the first location in the second graphical representation of data, a representation of a third subset of the second data set associated with the fourth variable; and
in accordance with a determination that the second input corresponds to the second location in the second graphical representation of data different from the first location, a representation of a fourth subset of the second data set that is associated with the fourth variable and that is different than the third subset of the second data set.

3. The computer system of claim 1, wherein displaying the plurality of user interface objects includes:
displaying the first user interface object positioned relative to the first graphical representation of data;
displaying the second user interface object positioned relative to the second graphical representation of data; and
displaying the first user interface object and the second user interface object having a same position along an axis.

4. The computer system of claim 1, wherein the second variable is the first variable.

5. The computer system of claim 1, wherein the first variable and the second variable are time-based variables.

6. The computer system of claim 1, wherein:
the representation of the first subset of the first data set includes a total value of the first subset of the first data set determined over a first subset of time;
the representation of the first subset of the second data set includes a total value of the first subset of the second data set determined over a second subset of time;
the first subset of time is determined based on a current time range corresponding to the first data set; and
the second subset of time is determined based on a current time range corresponding to the second data set.

7. The computer system of claim 1, wherein:
the representation of the first subset of the first data set includes an average value of the first subset of the first data set determined over a third subset of time; and
the representation of the first subset of the second data set includes an average value of the first subset of the second data set determined over a fourth subset of time.

8. The computer system of claim 1, the one or more programs further including instructions for:
detecting, via the one or more input devices, a third input corresponding to the first graphical representation of data; and
in response to detecting the third input; displaying:
a first graphical indication of a source associated with the first data set; and
a second graphical indication of a source associated with the second data set.

9. The computer system of claim 1, wherein:
the first data set corresponds to a fifth subset of time;
the second data set corresponds to a sixth subset of time;
the representation of the first subset of the first data set includes an indication of a first portion of the fifth subset of time during which a measured value of the first subset of the first data set exceeds a first predetermined threshold value; and
the representation of the first subset of the second data set includes an indication of a first portion of the sixth subset of time during which a measured value of the first subset of the second data set exceeds a second predetermined threshold value.

10. The computer system of claim 1, wherein the first variable and the second variable have a first value, the one or more programs further including instructions for:
detecting, via the one or more input devices, an input corresponding to a request to adjust a value of the first variable and the second variable; and
in response to detecting the input corresponding to a request to adjust the value of the first variable and the second variable:
in accordance with a determination that the input corresponds to a first request to adjust the first variable and the second variable, changing the first value of the first variable and the second variable to a second value different from the first value; and
in accordance with a determination that the input corresponds to a second request to adjust the first variable and the second variable, changing the first value of the first variable and the second variable to a third value different from the first value and the second value.

11. The computer system of claim 1, wherein the first user interface object is displayed having an initial position relative to the first graphical representation of data, and the second user interface object is displayed having an initial position relative to the second graphical representation of data, the one or more programs further including instructions for:
detecting, via the one or more input devices, a fourth input corresponding to the first graphical representation of data; and
in response to detecting the fourth input corresponding to the first graphical representation of data, updating display of the plurality of user interface objects, including:
displaying the first user interface object having an updated position relative to the first graphical representation of data and different from the initial position relative to the first graphical representation of data; and
displaying the second user interface object having an updated position relative to the second graphical representation of data and different from the initial position relative to the second graphical representation of data.

12. A non-transitory computer-readable storage medium storing one or more programs configured to be executed by one or more processors of a computer system that is in communication with a display generation component and one or more input devices, the one or more programs including instructions for:
displaying, via the display generation component, a plurality of graphical representations of data, including:
a first graphical representation of data corresponding to a first data set; and
a second graphical representation of data corresponding to a second data set that is different from the first data set;
detecting, via the one or more input devices, a first input corresponding to the first graphical representation of data; and
in response to detecting the first input corresponding to the first graphical representation of data, displaying a plurality of user interface objects, including:
a first user interface object associated with the first graphical representation of data and based on a first variable that is selected based on a location of the first input, wherein displaying the first user interface object includes replacing display of a portion of the first graphical representation of data with at least a portion of the first user interface object, and wherein the first user interface object includes:
in accordance with a determination that the first input corresponds to a first location in the first graphical representation of data, a representation of a first subset of the first data set associated with the first variable; and
in accordance with a determination that the first input corresponds to a second location in the first graphical representation of data different from the first location, a representation of a second subset of the first data set that is associated with the first variable and that is different than the first subset of the first data set; and
a second user interface object associated with the second graphical representation of data and based on a second variable that is selected based on a location of the first input, wherein displaying the second user interface object includes replacing display of a portion of the second graphical representation of data with at least a portion of the second user interface object, and wherein the second user interface object includes:
in accordance with a determination that the first input corresponds to the first location in the first graphical representation of data, a representation of a first subset of the second data set associated with the second variable; and
in accordance with a determination that the first input corresponds to the second location in the first graphical representation of data different from the first location, a representation of a second subset of the second data set that is associated with the second variable and that is different than the first subset of the second data set.

13. A method, comprising:
at a computer system that is in communication with a display generation component and one or more input devices:
displaying, via the display generation component, a plurality of graphical representations of data, including:
a first graphical representation of data corresponding to a first data set; and
a second graphical representation of data corresponding to a second data set that is different from the first data set;
detecting, via the one or more input devices, a first input corresponding to the first graphical representation of data; and
in response to detecting the first input corresponding to the first graphical representation of data, displaying a plurality of user interface objects, including:
a first user interface object associated with the first graphical representation of data and based on a first variable that is selected based on a location of the first input, wherein displaying the first user interface object includes replacing display of a portion of the first graphical representation of data with at least a portion of the first user interface object, and wherein the first user interface object includes:
in accordance with a determination that the first input corresponds to a first location in the first graphical representation of data, a representation of a first subset of the first data set associated with the first variable; and
in accordance with a determination that the first input corresponds to a second location in the first graphical representation of data different from the first location, a representation of a second subset of the first data set that is associated with the first variable and that is different than the first subset of the first data set; and
a second user interface object associated with the second graphical representation of data and based on a second variable that is selected based on a location of the first input, wherein displaying the second user interface object includes replacing display of a portion of the second graphical representation of data with at least a portion of the second user interface object, and wherein the second user interface object includes:
in accordance with a determination that the first input corresponds to the first location in the first graphical representation of data, a representation of a first subset of the second data set associated with the second variable; and
in accordance with a determination that the first input corresponds to the second location in the first graphical representation of data different from the first location, a representation of a second subset of the second data set that is associated with the second variable and that is different than the first subset of the second data set.
